SEQUENCE LISTING (125 sequences)

SEQ ID NO: 1 - DNA, 64 bp, synthetic construct
  source 1..64: mol_type = other DNA
agatcggaagagcacacgtctgaactccagtcacattatccctatagtgagtcgtattaaattc

SEQ ID NO: 2 - DNA, 57 bp, synthetic construct
  source 1..57: mol_type = other DNA
agatcggaagagcacacgtctgaactccagtcaccctatagtgagtcgtattagtca

SEQ ID NO: 3 - DNA, 38 bp, synthetic construct
  source 1..38: mol_type = other DNA
tgactaatacgactcactatagggtgactggagttcag

SEQ ID NO: 4 - DNA, 45 bp, synthetic construct
  source 1..45: mol_type = other DNA
gaatttaatacgactcactatagggataatgtgactggagttcag

SEQ ID NO: 5 - DNA, 30 bp, synthetic construct
  source 1..30: mol_type = other DNA
agatcggaagagcgtcgtgtagggaaagag

SEQ ID NO: 6 - DNA, 20 bp, synthetic construct
  source 1..20: mol_type = other DNA
taatacgactcactataggg

SEQ ID NO: 7 - DNA, 20 bp, synthetic construct
  source 1..20: mol_type = other DNA
ccctatagtgagtcgtatta

SEQ ID NO: 8 - DNA, 30 bp, synthetic construct
  source 1..30: mol_type = other DNA
gaatttaatacgactcactatagggataat

SEQ ID NO: 9 - DNA, 30 bp, synthetic construct
  source 1..30: mol_type = other DNA
attatccctatagtgagtcgtattaaattc

SEQ ID NO: 10 - DNA, 49 bp, synthetic construct
  source 1..49: mol_type = other DNA
aatgatacggcgaccaccgagatctacactctttccctacacgacgctc

SEQ ID NO: 11 - DNA, 52 bp, synthetic construct
  source 1..52: mol_type = other DNA
caagcagaagacggcatacgagatcgtgatgtgactggagttcagacgtgtg

SEQ ID NO: 12 - DNA, 68 bp, synthetic construct
  source 1..68: mol_type = other DNA
  modified_base 68: mod_base = OTHER; note = 2',3'-dideoxycytidine
  modified_base <1: mod_base = OTHER; note = Phosphorylation
nnnnnatcgtagatcggaagagcacacgtctgaactccagtcaccctatagtgagtcgtattagtcac

SEQ ID NO: 13 - DNA, 83 bp, synthetic construct
  source 1..83: mol_type = other DNA
  modified_base <1: mod_base = OTHER; note = Phosphorylation
  modified_base 83: mod_base = OTHER; note = Spacer C3
nnnnncatattcctggtggagatcggaagagcacacgtctgaactccagtcacattatccctatagtgagtcgtattaaattc

SEQ ID NO: 14 - DNA, 33 bp, synthetic construct
  source 1..33: mol_type = other DNA
  modified_base <1: mod_base = OTHER; note = Phosphorylation
  modified_base 33: mod_base = OTHER; note = 2',3'-dideoxycytidine
nnagatcggaagagcgtcgtgtagggaaagagc

SEQ ID NO: 15 - DNA, 20 bp, synthetic construct
  source 1..20: mol_type = other DNA
agatcggagcacacgtctga

SEQ ID NO: 16 - DNA, 83 bp, synthetic construct
  source 1..83: mol_type = other DNA
  modified_base <1: mod_base = OTHER; note = Adenylation
nnnnnnnnnnnnnnnnnnnagatcggaagagcacacgtctgaactccagtcacattatccctatagtgagtcgtattaaattc

SEQ ID NO: 17 - RNA, 61 nt, synthetic construct
  source 1..61: mol_type = other RNA
  modified_base <1: mod_base = OTHER; note = 5' triphosphate
gggataatgtgactggagttcagacgtgtgctcttccgatctnnnnnnnnnnnnnnnnnnn

SEQ ID NO: 18 - DNA, 60 bp, synthetic construct
  source 1..60: mol_type = other DNA
aatgatacggcgaccaccgagatctacactctttccctacacgacgctcttccgatctnn

SEQ ID NO: 19 - DNA, 33 bp, synthetic construct
  source 1..33: mol_type = other DNA
acactctttccctacacgacgctcttccgatct

SEQ ID NO: 20 - DNA, 33 bp, synthetic construct
  source 1..33: mol_type = other DNA
gtgactggagttcagacgtgtgctcttccgatc

SEQ ID NO: 21 - DNA, 60 bp, synthetic construct; identical to SEQ ID NO: 18
  source 1..60: mol_type = other DNA

SEQ ID NO: 22 - DNA, 60 bp, synthetic construct
  source 1..60: mol_type = other DNA
nnagatcggaagagcgtcgtgtagggaaagagtgtagatctcggtggtcgccgtatcatt

SEQ ID NO: 23 - DNA, 90 bp, synthetic construct
  source 1..90: mol_type = other DNA
nnnnnnnnnnnnnnnnnnnnnnnnagatcggaagagcacacgtctgaactccagtcacgatctcgtatctcgtatgccgtcttctgcttg

SEQ ID NO: 24 - DNA, 90 bp, synthetic construct
  source 1..90: mol_type = other DNA
caagcagaagacggcatacgagatacgagatcgtgactggagttcagacgtgtgctcttccgatctnnnnnnnnnnnnnnnnnnnnnnnn

SEQ ID NO: 25 - DNA, 29 bp, synthetic construct
  source 1..29: mol_type = other DNA
aatgatacggcgaccaccgagatctacac

SEQ ID NO: 26 - DNA, 29 bp, synthetic construct
  source 1..29: mol_type = other DNA
gtgtagatctcggtggtcgccgtatcatt

SEQ ID NO: 27 - DNA, 21 bp, synthetic construct
  source 1..21: mol_type = other DNA
caagcagaagacggcatacga

SEQ ID NO: 28 - DNA, 21 bp, synthetic construct
  source 1..21: mol_type = other DNA
tcgtatgccgtcttctgcttg

SEQ ID NO: 29 - DNA, 49 bp, synthetic construct; identical to SEQ ID NO: 10
  source 1..49: mol_type = other DNA
  modified_base <1: mod_base = OTHER; note = Phosphorylation

SEQ ID NO: 30 - DNA, 14 bp, synthetic construct
  source 1..14: mol_type = other DNA
catattcctggtgg

SEQ ID NO: 31 - DNA, 14 bp, synthetic construct
  source 1..14: mol_type = other DNA
acaacaaggttcgg

SEQ ID NO: 32 - DNA, 14 bp, synthetic construct
  source 1..14: mol_type = other DNA
tcggtgaggattgg

SEQ ID NO: 33 - DNA, 14 bp, synthetic construct
  source 1..14: mol_type = other DNA
gacaacatcagcgg

SEQ ID NO: 34 - DNA, 14 bp, synthetic construct
  source 1..14: mol_type = other DNA
atattgcaacctgg

SEQ ID NO: 35 - DNA, 14 bp, synthetic construct
  source 1..14: mol_type = other DNA
ttggaaccttgccg

SEQ ID NO: 36 - DNA, 14 bp, synthetic construct
  source 1..14: mol_type = other DNA
ccttctccatcctg

SEQ ID NO: 37 - DNA, 14 bp, synthetic construct
  source 1..14: mol_type = other DNA
cgcgaattgattag

SEQ ID NO: 38 - DNA, 14 bp, synthetic construct
  source 1..14: mol_type = other DNA
ttaggcgttggtgg

SEQ ID NO: 39 - DNA, 14 bp, synthetic construct
  source 1..14: mol_type = other DNA
acaagtctcacgtg

SEQ ID NO: 40 - DNA, 14 bp, synthetic construct
  source 1..14: mol_type = other DNA
agacggagttgtgg

SEQ ID NO: 41 - DNA, 14 bp, synthetic construct
  source 1..14: mol_type = other DNA
acatgagtgttctg

SEQ ID NO: 42 - DNA, 14 bp, synthetic construct
  source 1..14: mol_type = other DNA
atatgaaggcttgg

SEQ ID NO: 43 - DNA, 14 bp, synthetic construct
  source 1..14: mol_type = other DNA
ccttagatcctccg

SEQ ID NO: 44 - DNA, 14 bp, synthetic construct
  source 1..14: mol_type = other DNA
tacataagccttcg

SEQ ID NO: 45 - DNA, 14 bp, synthetic construct
  source 1..14: mol_type = other DNA
gccaggaagtcttg

SEQ ID NO: 46 - DNA, 14 bp, synthetic construct
  source 1..14: mol_type = other DNA
agcaagactagcgg

SEQ ID NO: 47 - DNA, 14 bp, synthetic construct
  source 1..14: mol_type = other DNA
agccttggatctgg

SEQ ID NO: 48 - DNA, 14 bp, synthetic construct
  source 1..14: mol_type = other DNA
gaggcttaacttgg

SEQ ID NO: 49 - DNA, 14 bp, synthetic construct
  source 1..14: mol_type = other DNA
tgacaggacttcgg

SEQ ID NO: 50 - DNA, 14 bp, synthetic construct
  source 1..14: mol_type = other DNA
ggagcactagcttg

SEQ ID NO: 51 - DNA, 14 bp, synthetic construct
  source 1..14: mol_type = other DNA
atgttatctcgtgg

SEQ ID NO: 52 - DNA, 14 bp, synthetic construct
  source 1..14: mol_type = other DNA
agctaatgttgtcg

SEQ ID NO: 53 - DNA, 14 bp, synthetic construct
  source 1..14: mol_type = other DNA
ggaacgatattcag

SEQ ID NO: 54 - DNA, 14 bp, synthetic construct
  source 1..14: mol_type = other DNA
tgctgtcagattgc

SEQ ID NO: 55 - DNA, 14 bp, synthetic construct
  source 1..14: mol_type = other DNA
ctattgccttcgcc

SEQ ID NO: 56 - DNA, 14 bp, synthetic construct
  source 1..14: mol_type = other DNA
cagaggctgttgtc

SEQ ID NO: 57 - DNA, 14 bp, synthetic construct
  source 1..14: mol_type = other DNA
atgagtagaccttc

SEQ ID NO: 58 - DNA, 14 bp, synthetic construct
  source 1..14: mol_type = other DNA
accgttaagtcgcc

SEQ ID NO: 59 - DNA, 14 bp, synthetic construct
  source 1..14: mol_type = other DNA
atctgtggttctac

SEQ ID NO: 60 - DNA, 14 bp, synthetic construct
  source 1..14: mol_type = other DNA
tgtgcggttcgttc

SEQ ID NO: 61 - DNA, 14 bp, synthetic construct
  source 1..14: mol_type = other DNA
aagcgccatggttc

SEQ ID NO: 62 - DNA, 14 bp, synthetic construct
  source 1..14: mol_type = other DNA
tcagaggtagttac

SEQ ID NO: 63 - DNA, 14 bp, synthetic construct
  source 1..14: mol_type = other DNA
gttggttagtcttc

SEQ ID NO: 64 - DNA, 14 bp, synthetic construct
  source 1..14: mol_type = other DNA
cattactataccgc

SEQ ID NO: 65 - DNA, 14 bp, synthetic construct
  source 1..14: mol_type = other DNA
cgacaactactgcc

SEQ ID NO: 66 - DNA, 14 bp, synthetic construct
  source 1..14: mol_type = other DNA
aaccacgataggcc

SEQ ID NO: 67 - DNA, 14 bp, synthetic construct
  source 1..14: mol_type = other DNA
gctagagactcttc

SEQ ID NO: 68 - DNA, 14 bp, synthetic construct
  source 1..14: mol_type = other DNA
gttaggaagttcac

SEQ ID NO: 69 - DNA, 14 bp, synthetic construct
  source 1..14: mol_type = other DNA
ttgatccgttcttc

SEQ ID NO: 70 - DNA, 14 bp, synthetic construct
  source 1..14: mol_type = other DNA
ggttagtagattcc

SEQ ID NO: 71 - DNA, 14 bp, synthetic construct
  source 1..14: mol_type = other DNA
ccggactaaccttc

SEQ ID NO: 72 - DNA, 14 bp, synthetic construct
  source 1..14: mol_type = other DNA
agcatggactctcc

SEQ ID NO: 73 - DNA, 14 bp, synthetic construct
  source 1..14: mol_type = other DNA
ttccagataccttc

SEQ ID NO: 74 - DNA, 14 bp, synthetic construct
  source 1..14: mol_type = other DNA
ataagacactctgc

SEQ ID NO: 75 - DNA, 14 bp, synthetic construct
  source 1..14: mol_type = other DNA
gaattctcgattcc

SEQ ID NO: 76 - DNA, 14 bp, synthetic construct
  source 1..14: mol_type = other DNA
gtcaattcacctac

SEQ ID NO: 77 - DNA, 14 bp, synthetic construct
  source 1..14: mol_type = other DNA
ggtattaagacgtc

SEQ ID NO: 78 - DNA, 14 bp, synthetic construct
  source 1..14: mol_type = other DNA
aatatcttcggcca

SEQ ID NO: 79 - DNA, 14 bp, synthetic construct
  source 1..14: mol_type = other DNA
agtggcgttaacaa

SEQ ID NO: 80 - DNA, 14 bp, synthetic construct
  source 1..14: mol_type = other DNA
gtgtccggcattga

SEQ ID NO: 81 - DNA, 14 bp, synthetic construct
  source 1..14: mol_type = other DNA
caaccggagaagaa

SEQ ID NO: 82 - DNA, 14 bp, synthetic construct
  source 1..14: mol_type = other DNA
ctcaggacttagta

SEQ ID NO: 83 - DNA, 14 bp, synthetic construct
  source 1..14: mol_type = other DNA
gcttagactagcta

SEQ ID NO: 84 - DNA, 14 bp, synthetic construct
  source 1..14: mol_type = other DNA
acattgtccaccaa

SEQ ID NO: 85 - DNA, 14 bp, synthetic construct
  source 1..14: mol_type = other DNA
atcgcgccaaccaa

SEQ ID NO: 86 - DNA, 14 bp, synthetic construct
  source 1..14: mol_type = other DNA
gcgaagtccgagaa

SEQ ID NO: 87 - DNA, 14 bp, synthetic construct
  source 1..14: mol_type = other DNA
attaggagctcgaa

SEQ ID NO: 88 - DNA, 14 bp, synthetic construct
  source 1..14: mol_type = other DNA
gcgtgacgcagtaa

SEQ ID NO: 89 - DNA, 14 bp, synthetic construct
  source 1..14: mol_type = other DNA
ggatccttagacaa

SEQ ID NO: 90 - DNA, 14 bp, synthetic construct
  source 1..14: mol_type = other DNA
gaagcataggagaa

SEQ ID NO: 91 - DNA, 14 bp, synthetic construct
  source 1..14: mol_type = other DNA
cacctccatgtgta

SEQ ID NO: 92 - DNA, 14 bp, synthetic construct
  source 1..14: mol_type = other DNA
cgcatgctatagaa

SEQ ID NO: 93 - DNA, 14 bp, synthetic construct
  source 1..14: mol_type = other DNA
tggtcgatcttgaa

SEQ ID NO: 94 - DNA, 14 bp, synthetic construct
  source 1..14: mol_type = other DNA
tagatctcgcagaa

SEQ ID NO: 95 - DNA, 14 bp, synthetic construct
  source 1..14: mol_type = other DNA
tagtgagctagtca

SEQ ID NO: 96 - DNA, 14 bp, synthetic construct
  source 1..14: mol_type = other DNA
atgagcgcatatca

SEQ ID NO: 97 - DNA, 14 bp, synthetic construct
  source 1..14: mol_type = other DNA
ctatacagtgcgta

SEQ ID NO: 98 - DNA, 14 bp, synthetic construct
  source 1..14: mol_type = other DNA
gagagaaggttgta

SEQ ID NO: 99 - DNA, 14 bp, synthetic construct
  source 1..14: mol_type = other DNA
ctggagaggtacaa

SEQ ID NO: 100 - DNA, 14 bp, synthetic construct
  source 1..14: mol_type = other DNA
ttatacgatcgcca

SEQ ID NO: 101 - DNA, 14 bp, synthetic construct
  source 1..14: mol_type = other DNA
ctcgaatcctacaa

SEQ ID NO: 102 - DNA, 14 bp, synthetic construct
  source 1..14: mol_type = other DNA
aacagaccgttaca

SEQ ID NO: 103 - DNA, 14 bp, synthetic construct
  source 1..14: mol_type = other DNA
gtgcctcaccgata

SEQ ID NO: 104 - DNA, 14 bp, synthetic construct
  source 1..14: mol_type = other DNA
ttgcgcctcacata

SEQ ID NO: 105 - DNA, 14 bp, synthetic construct
  source 1..14: mol_type = other DNA
accgcgaagatata

SEQ ID NO: 106 - DNA, 14 bp, synthetic construct
  source 1..14: mol_type = other DNA
caggaccgtatata

SEQ ID NO: 107 - DNA, 14 bp, synthetic construct
  source 1..14: mol_type = other DNA
ttatctagacgagg

SEQ ID NO: 108 - DNA, 14 bp, synthetic construct
  source 1..14: mol_type = other DNA
actatcgcgctagg

SEQ ID NO: 109 - DNA, 14 bp, synthetic construct
  source 1..14: mol_type = other DNA
attggagcattagg

SEQ ID NO: 110 - DNA, 14 bp, synthetic construct
  source 1..14: mol_type = other DNA
atgcagagctgacg

SEQ ID NO: 111 - DNA, 14 bp, synthetic construct
  source 1..14: mol_type = other DNA
attgtatcaccacg

SEQ ID NO: 112 - DNA, 14 bp, synthetic construct
  source 1..14: mol_type = other DNA
gtaagtcctatacg

SEQ ID NO: 113 - DNA, 14 bp, synthetic construct
  source 1..14: mol_type = other DNA
ttcatgaccagatg

SEQ ID NO: 114 - DNA, 14 bp, synthetic construct
  source 1..14: mol_type = other DNA
gacaggagagagat

SEQ ID NO: 115 - DNA, 14 bp, synthetic construct
  source 1..14: mol_type = other DNA
ccagtccaacagat

SEQ ID NO: 116 - DNA, 14 bp, synthetic construct
  source 1..14: mol_type = other DNA
cagaatggacagat

SEQ ID NO: 117 - DNA, 14 bp, synthetic construct
  source 1..14: mol_type = other DNA
gttcgcaggcagat

SEQ ID NO: 118 - DNA, 14 bp, synthetic construct
  source 1..14: mol_type = other DNA
catgcgagtcggat

SEQ ID NO: 119 - DNA, 14 bp, synthetic construct
  source 1..14: mol_type = other DNA
ggaacgaggacgat

SEQ ID NO: 120 - DNA, 14 bp, synthetic construct
  source 1..14: mol_type = other DNA
cggaagccacttat

SEQ ID NO: 121 - DNA, 14 bp, synthetic construct
  source 1..14: mol_type = other DNA
agtccactaggcgt

SEQ ID NO: 122 - DNA, 14 bp, synthetic construct
  source 1..14: mol_type = other DNA
ttggtctggtgcgt

SEQ ID NO: 123 - DNA, 14 bp, synthetic construct
  source 1..14: mol_type = other DNA
ctcaccgtgatcgt

SEQ ID NO: 124 - DNA, 14 bp, synthetic construct
  source 1..14: mol_type = other DNA
taacaagtgctcgt

SEQ ID NO: 125 - DNA, 14 bp, synthetic construct
  source 1..14: mol_type = other DNA
ggattccagttcgt

(19) United States
(12) Patent Application Publication
(10) Pub. No.: US 2026/0258404 A1
(43) Pub. Date: Sep. 3, 2026

(54) METHOD FOR CREATING DNA LIBRARY USING RNA TEMPLATE RNA AMPLIFICATION

(71) Applicant: RIKEN, Saitama (JP)

(72) Inventors: Shintaro IWASAKI, Saitama (JP); Yuichi SHICHINO, Saitama (JP); Mari MITO, Saitama (JP); Taisei WAKIGAWA, Saitama (JP)

(73) Assignee: RIKEN, Saitama (JP)

(21) Appl. No.: 19/163,984

(22) PCT Filed: Mar. 12, 2024
(86) PCT No.: PCT/JP2024/009605

§ 371 Date: Sep. 10, 2025

(30) Foreign Application Priority Data
Mar. 13, 2023 (JP) 2023-038505

(51) Int. Cl. C12N 15/10; C12Q 1/6855; C12Q 1/686; C12Q 1/6869

(52) U.S. Cl. CPC C12N 15/1096; C12N 15/1093; C12Q 1/6855; C12Q 1/686; C12Q 1/6869

(57) ABSTRACT

A method for preparing a DNA library from a sample containing RNA includes ligating a first DNA linker to the 3′ end of at least one RNA molecule in a sample containing RNA to obtain an RNA molecule having the first DNA linker, hybridizing a first oligonucleotide with the RNA molecule having the first DNA linker to obtain the RNA molecule having a double-stranded DNA promoter region, obtaining a complementary strand of the RNA molecule having the first DNA linker (complementary RNA) by in vitro transcription using the RNA molecule having the double-stranded DNA promoter region as a template and an RNA polymerase that recognizes the promoter sequence and initiates transcription; and ligating a second DNA linker to the 3′ end of the complementary RNA and further obtaining a single-stranded DNA by reverse transcription using the complementary RNA having the second DNA linker as a template.

### TECHNICAL FIELD

The present invention relates to a method for preparing a DNA library from a sample containing RNA. The present invention further relates to a method for sequencing and/or quantifying a RNA molecule in a sample containing RNA.

### CROSS REFERENCE TO RELATED APPLICATION

This application claims conventional priority based on Japanese Patent Application No. 2023-038505, filed in Japan on Mar. 13, 2023, the entire description of which is hereby particularly incorporated by reference as a disclosure.

### BACKGROUND ART

While transcriptome analysis has been recognized as a proxy for gene expression analysis, recent studies have shown that regulation at the protein synthesis level has a significant impact on the final output of genes. Ribosome profiling (also referred to as Ribo-Seq in this description), a technique based on RNase footprinting of mRNA by ribosomes and subsequent deep sequencing, has emerged as a revolutionary approach to study protein synthesis in cells (Non-patent document 1). The applications of Ribo-Seq technique spread over an extremely wide range covering, for example, measurement of translation efficiency of the entire transcriptomes (Non-patent documents 2 and 3), de novo assignment of open reading frames (ORFs) (Non-patent document 4), exploration of ribosome movement speed at codon-level resolution (Non-patent documents 2 and 3), estimation of ribosome structure (Non-patent documents 5 and 6), and so forth. The widespread application of Ribo-Seq technique for a variety of materials and under a variety of biological conditions has revealed the hierarchical structure of the regulation of protein synthesis, which has not been revealed before. Ribo-Seq is also expected to be applied to rare, valuable, and difficult-to-prepare samples. However, the standard protocols thereof require substantial amounts of cells for high-quality library preparation (Non-patent documents 7 and 8), technical hurdles exist for low inputs (i.e., small amounts of RNA material). Therefore, there is a challenge to optimize Ribo-Seq for small amounts of RNA material.

The one-pot reaction (meaning multi-step reactions by feeding reactants sequentially into a reaction vessel) for library preparation is an advantageous approach to avoid sample loss. For this purpose, ligation-free methods can be applied to RNA molecules, e.g., isolated ribosome footprints. This system sequentially performs reactions for template-switched 3′ end poly (A) tailing, reverse transcription, and linker addition in the same tube (Non-patent document 9 to 12). The recently reported single cell ribosome profiling (sCRibo-Seq) technique initiates the one-pot reaction from cell lysate, followed by RNase digestion, first linker ligation, reverse transcription, second linker ligation, and PCR (Non-patent document 13). However, in these approaches, there was no means to amplify DNA or RNA prior to PCR, and therefore there was a risk of losing material during the procedure.

### PRIOR ART REFERENCES

### Non-Patent Documents

Non-patent document 1: Ingolia, N. T., Ghaemmaghami, S., Newman, J. R. & Weissman, J. S. Genomewide analysis in vivo of translation with nucleotide resolution using ribosome profiling. Science 324, 218-223 (2009).
Non-patent document 2: Brar, G. A. & Weissman, J. S. Ribosome profiling reveals the what, when, where and how of protein synthesis. Nat. Rev. Mol. Cell Biol. 16, 651-664 (2015).
Non-patent document 3: Ingolia, N. T., Hussmann, J. A. & Weissman, J. S. Ribosome profiling: global views of translation. Cold Spring Harb. Perspect. Biol. 11, a032698 (2019).
Non-patent document 4: Calviello, L. & Ohler, U. Beyond read-counts: ribo-seq data analysis to understand the functions of the transcriptome. Trends Genet. 33, 728-744 (2017).
Non-patent document 5: Lareau, L. F., Hite, D. H., Hogan, G. J. & Brown, P. O. Distinct stages of the translation elongation cycle revealed by sequencing ribosome-protected mRNA fragments. Elife 3, e01257 (2014).
Non-patent document 6: Wu, C. C., Zinshteyn, B., Wehner, K. A. & Green, R. High-resolution ribosome profiling defines discrete ribosome elongation states and translational regulation during cellular stress. Mol. Cell 73, 959-970.e5 (2019).
Non-patent document 7: McGlincy, N. J. & Ingolia, N. T. Transcriptome-wide measurement of translation by ribosome profiling. Methods 126, 112-129 (2017).
Non-patent document 8: Mito, M., Mishima, Y. & Iwasaki, S. Protocol for disome profiling to survey ribosome collision in humans and zebrafish. STAR Protoc 1, 100168 (2020).
Non-patent document 9: Hornstein, N. et al. Ligation-free ribosome profiling of cell type-specific translation in the brain. Genome Biol. 17, 149 (2016).
Non-patent document 10: Li, Q., Yang, H., Stroup, E. K., Wang, H. & Ji, Z. Low-input RNase footprinting for simultaneous quantification of cytosolic and mitochondrial translation. Genome Res. 32, 545-557 (2022).
Non-patent document 11: Xiong, Z. et al. Ultrasensitive Ribo-seq reveals translational landscapes during mammalian oocyte-to-embryo transition and pre-implantation development. Nat. Cell Biol. 24, 968-980 (2022).
Non-patent document 12: Zou, Z. et al. Translatome and transcriptome co-profiling reveals a role of TPRXs in human zygotic genome activation. Science 378, abo7923 (2022).
Non-patent document 13: VanInsberghe, M., van den Berg, J., Andersson-Rolf, A., Clevers, H. & van Oudenaarden, A. Single-cell Ribo-seq reveals cell cycle-dependent translational pausing. Nature 597, 561-565 (2021).

### SUMMARY OF THE INVENTION

### Object to be Achieved by the Invention

An object of the present invention is to provide a method for creating a DNA library in a procedure with minimal loss of RNA material by amplifying RNA from RNA material at an early stage of the procedure, and a method for sequencing and/or quantifying RNA molecules using this DNA library.

### Means for Achieving the Object

As a result of diligent researches to achieve the above object, the inventors of the present invention have developed a method for creating a DNA library from a sample containing RNA, especially a small amount of RNA material, by RNA amplification with RNA polymerase, which can use an RNA molecule having a DNA linker as a template for in vitro transcription, and a method for sequencing and/or quantifying RNA molecules. The linker oligonucleotide, rationally designed by the inventors of the present invention, maximizes linear amplification of a small amount of RNA material, which is less biased than exponential amplification by PCR. The method of the present invention is widely applicable to translation studies not only when small amounts of material are available, but also when standard amounts of material can be secured.

The invention provides the following inventions.

<1>

A method for preparing a DNA library from a sample containing RNA, comprising:
(1) the step of ligating a first DNA linker to the 3′ end of at least one RNA molecule in a sample containing RNA to obtain an RNA molecule having the first DNA linker, wherein the first DNA linker comprises at least a promoter antisense sequence;
(2) the step of hybridizing a first oligonucleotide with the RNA molecule having the first DNA linker to obtain the RNA molecule having a double-stranded DNA promoter region, wherein the first oligonucleotide comprises a promoter sense sequence;
(3) the step of obtaining a complementary strand of the RNA molecule having the first DNA linker (complementary RNA) by in vitro transcription using the RNA molecule having the double-stranded DNA promoter region as a template and an RNA polymerase that recognizes the promoter sequence and initiates transcription; and
(4) the step of ligating a second DNA linker to the 3′ end of the complementary RNA and further obtaining a single-stranded DNA by reverse transcription using the complementary RNA having the second DNA linker as a template.

2> The method according to <1>, which further comprises (5) the step of obtaining a double-stranded DNA by PCR using the single-stranded DNA obtained in the step (4) as a template.

<3>

The method according to <1> or <2>, wherein the RNA polymerase used in the step (3) is T7 RNA polymerase.

<4>

The method according to any one of <1> to <3>, wherein the first DNA linker and the first oligonucleotide contain a sequence that increases transcription efficiency in a flanking region of the promoter sequence.

<5>

The method according to any one of <1> to <4>, wherein the DNA library is a next generation sequencing cDNA library.

<6>

The method according to any one of <1> to <5>, wherein the sample containing RNA is a lysate of less than 1×10⁶ cells.

<7>

The method according to any one of <1> to <6>, which is performed to generate a cDNA library for ribosome profiling.

<8>

The method according to <7>, wherein the at least one RNA molecule in the sample containing RNA is a ribosome footprint, the 3′ end of which is dephosphorylated prior to the step (1).

<9>

A method for sequencing and/or quantifying an RNA molecule in a sample containing RNA, comprising
the step of performing the steps (1) to (4) described in <1> and optionally further performing the step (5) described in <2> to prepare a DNA library; and
the step of analyzing the DNA library by a next-generation sequencing method to obtain data of sequencing and/or quantification of the RNA molecule in the sample containing RNA.

<10>

The method according to any one of <1> to <9>, which is for analyzing multiple samples using a multi-well plate.

<11>

The method according to any one of <1> to <10>, which is for analyzing mitochondrial translation.

<12>

A kit for performing the method according to any one of <1> to <11>, comprising
a first DNA linker comprising at least a promoter antisense sequence,
a first oligonucleotide comprising at least a promoter sense sequence; and
a second DNA linker.

<13>

The kit according to <12>, which further comprises an RNA polymerase that recognizes the promoter sequence; and/or
a reverse transcriptase; and/or
a second oligonucleotide used as a primer for reverse transcription; and/or
a pair of primer oligonucleotides for PCR.

<14>

The kit according to <12> or <13>, wherein the first DNA linker comprises any of the following nucleotide sequences:

(SEQ ID NO: 1)
AGATCGGAAGAGCACACGTCTGAACTCCAGTCACATTATCCCTATA
GTGAGTCGTATTAAATTC
and
(SEQ ID NO: 2)
AGATCGGAAGAGCACACGTCTGAACTCCAGTCACCCTATAGTGA
GTCGTATTAGTCA.

<15>

The kit according to any one of <12> to <14>, wherein the first oligonucleotide comprises any of the following nucleotide sequences:

(SEQ ID NO: 3)
TGACTAATACGACTCACTATAGGGTGACTGGAGTTCAG
and
(SEQ ID NO: 4)
GAATTTAATACGACTCACTATAGGGATAATGTGACTGGAGTTCAG.

<16>

The kit according to any one of <12> to <15>, wherein the second DNA linker comprises the following nucleotide sequence;

(SEQ ID NO: 5)
AGATCGGAAGAGCGTCGTGTAGGGAAAGAG.

<17>

The kit according to any one of <12> to <16>, wherein the first DNA linker and/or the second DNA linker further comprises a sequence for a unique molecular identifier (UMI) and/or a sample identifier sequence.

### BRIEF DESCRIPTION OF THE DRAWINGS

[FIG. 1-1] Outline of the steps of Standard Ribo-Seq (A) and Thor-Ribo-Seq (B) library preparation schemes.

[FIG. 1-2] (C) Gel photograph of the results obtained by adding the described amounts of initial materials to a PCR-amplified DNA library prepared by Standard Ribo-Seq method. The limitations of Standard Ribo-Seq method for small amounts of RNA material are shown. Random sequences were used as UMI. [FIG. 2-1] The translational status of small amounts of RNA material is evaluated by Thor-Ribo-Seq according to the present invention. (A) A schematic view of Thor-Ribo-Seq library creation strategy. RT, reverse transcription. The symbol “~” used in the drawing means about (approximately).

[FIG. 2-2] (B) A graph showing correspondence of the numbers of reads on transcripts from Standard Ribo-Seq and Thor-Ribo-Seq in the case of normal input (normal amount of RNA material). (C) A graph showing correspondence of average ribosome occupancies on the A-site codon sequence in the standard Ribo-Seq and Thor-Ribo-Seq data in the case of normal input. (D) Graphs showing correspondence of the numbers of reads on transcripts from Thor-Ribo-Seq with normal input and the “aliquoted-then-digested” experiment with low input (small amount of RNA material). (E) Graphs showing correspondence of average ribosome occupancies on the A-site codon sequence from Thor-Ribo-Seq with normal input and the “aliquoted-then-digested” experiment with low input. r, Pearson's correlation coefficient. The symbol “~” used in the graphs means about (approximately).

FIG. 3 Characteristics of ribosome footprints obtained by Thor-Ribo-Seq with normal input (normal amount of RNA material). (A) Distribution of footprint lengths from Standard Ribo-Seq and Thor-Ribo-Seq with normal input (normal amount of RNA material). (B) and (C) Metagene plots of the 5′ end positions of the ribosome footprints around the start codon in the experiments indicated. X-axis, position relative to the start codon (0 is the first nucleotide of the start codon); Y-axis, footprint length; color scale, read abundance; rpm, number of reads per million. (D) Number of genes detected in 4 million footprint reads (3 reads or more) in the experiments indicated. Representative transcripts for each gene as defined by MANE Select (Ensembl) were used. The symbol “~” used in the graph means about (approximately).

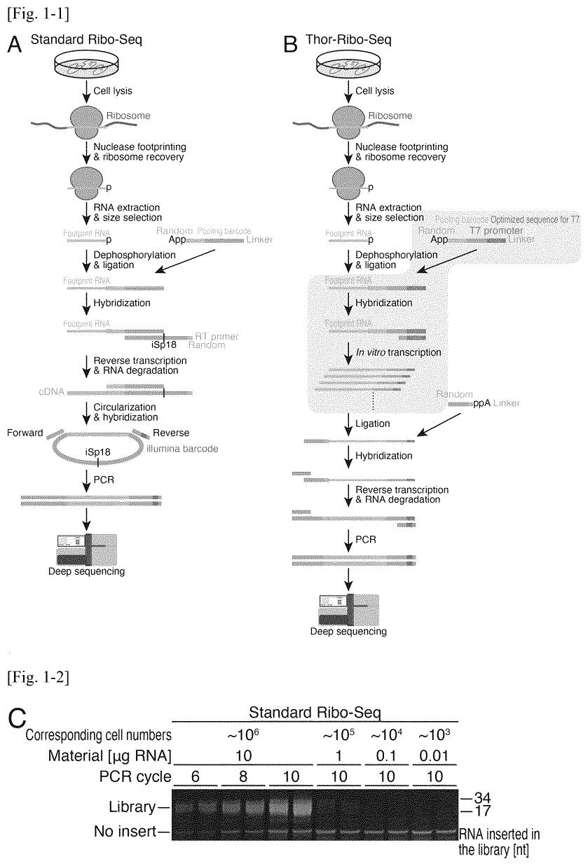

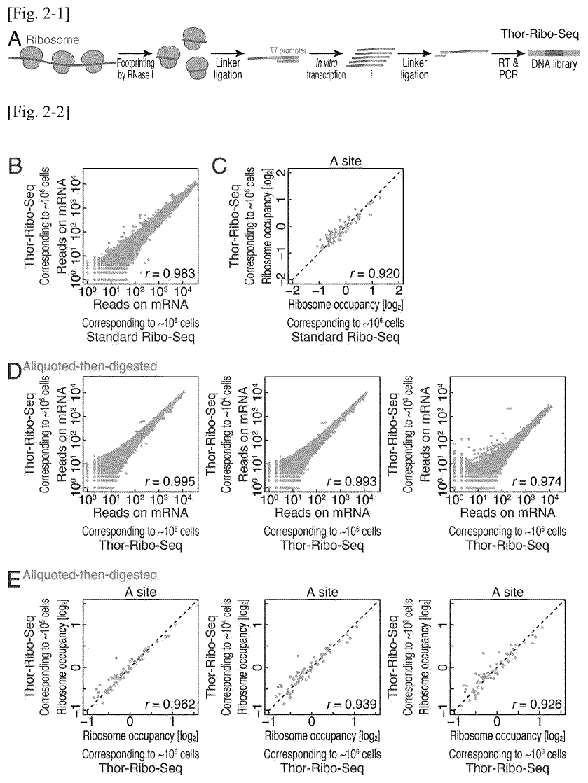

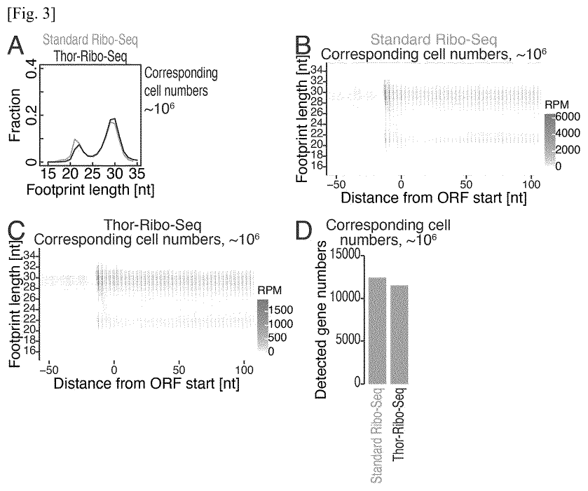

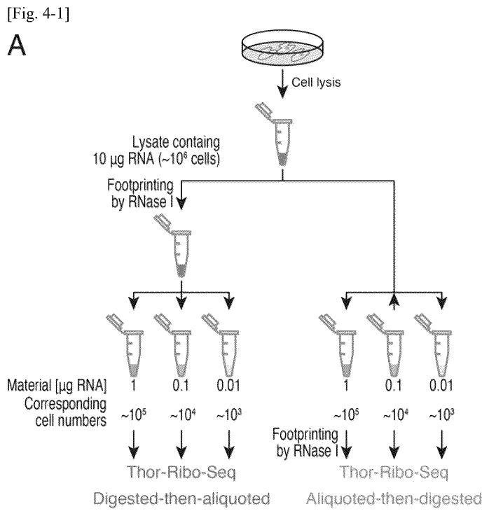

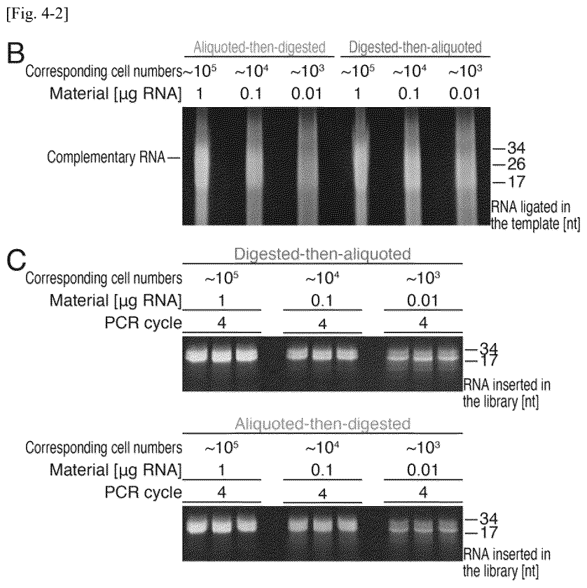

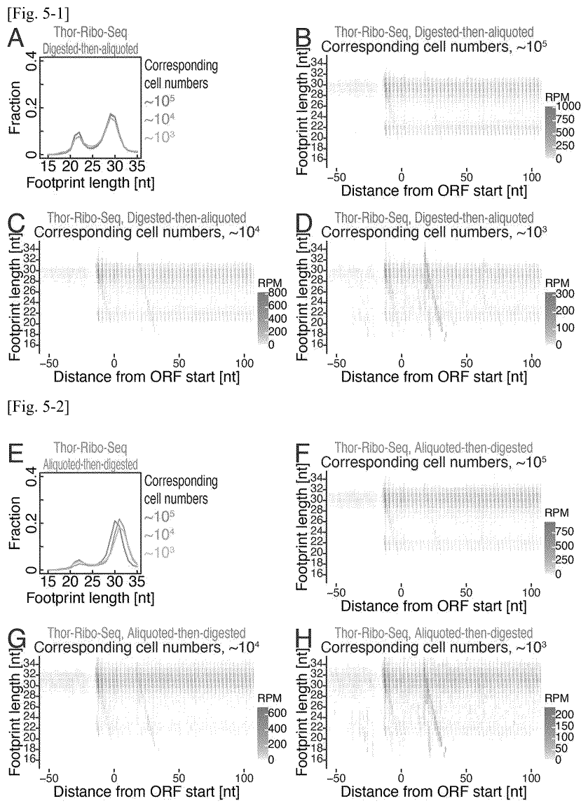

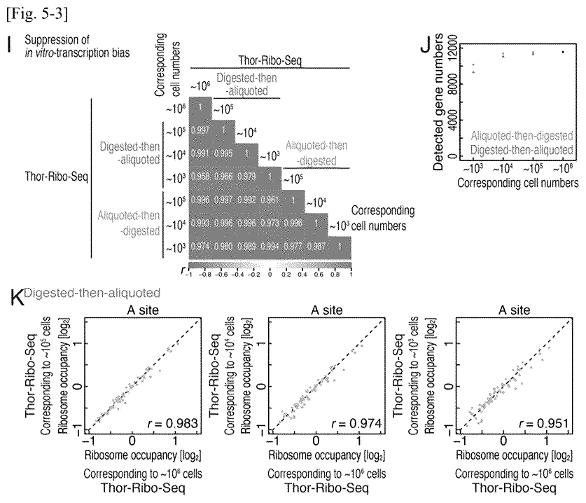

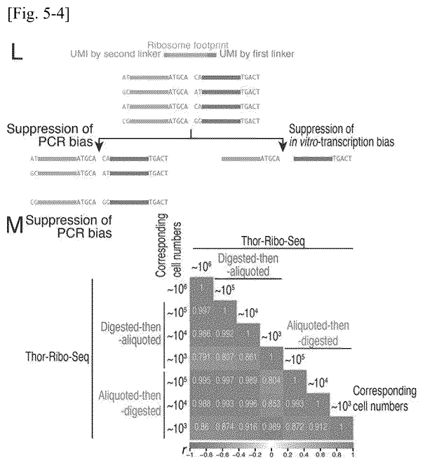

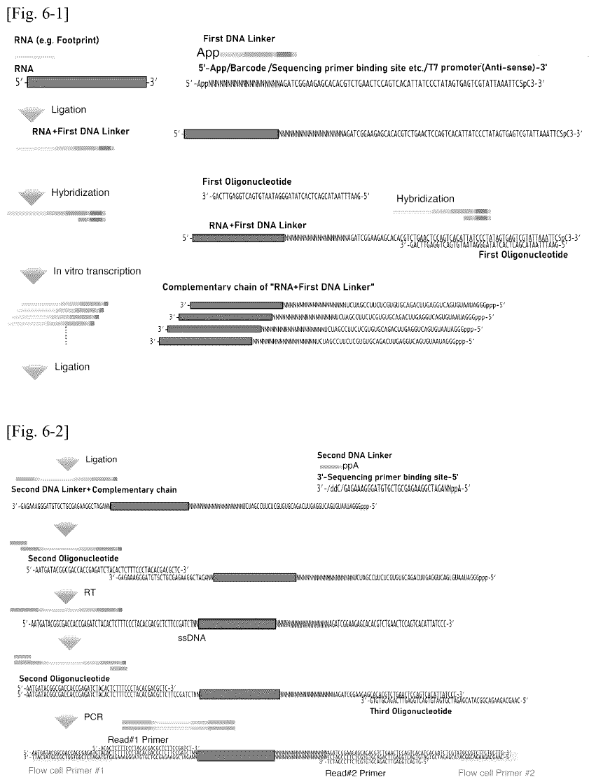

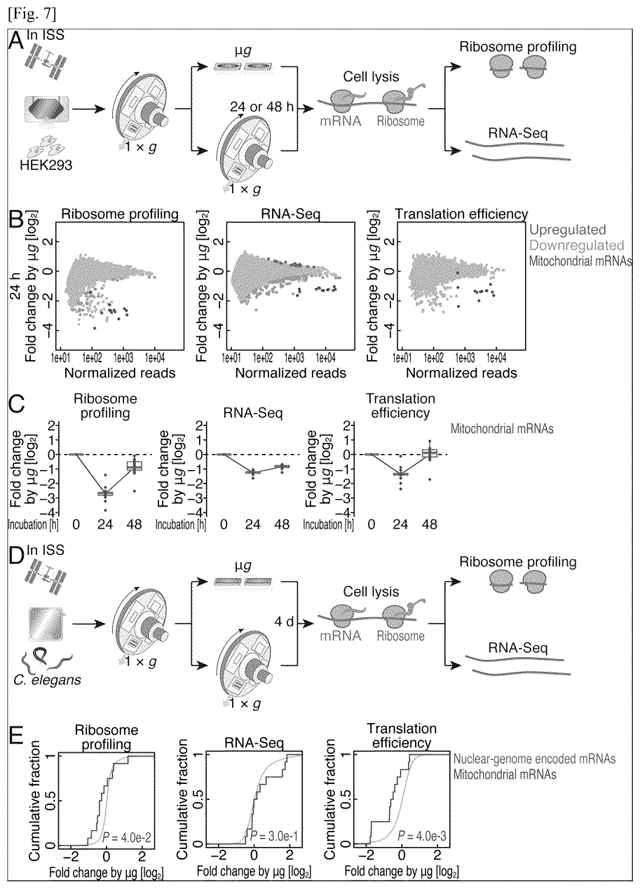

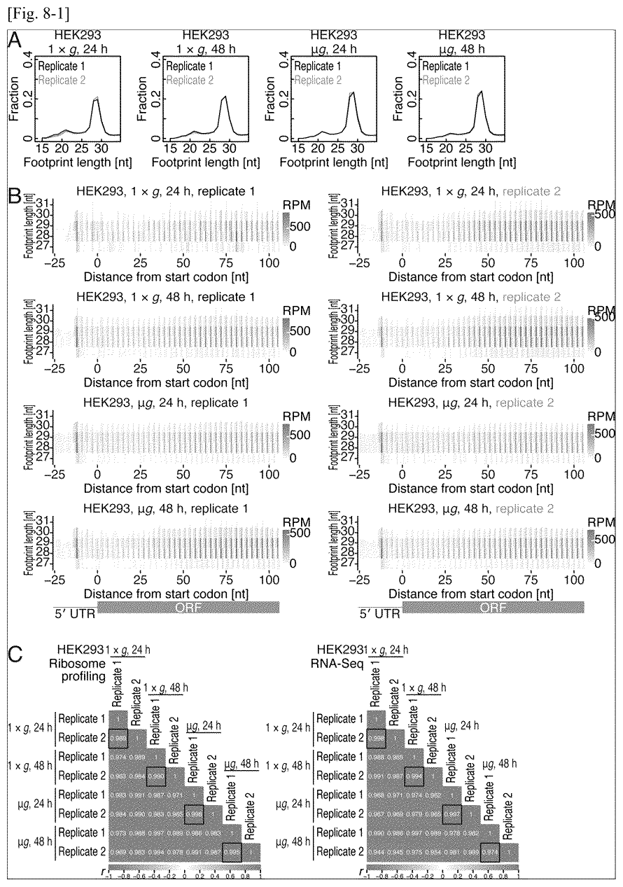

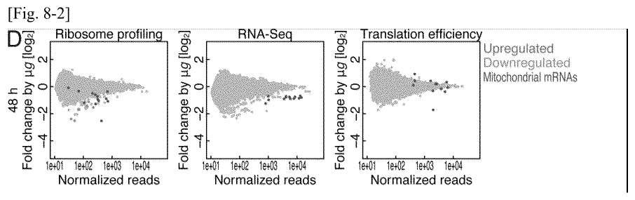

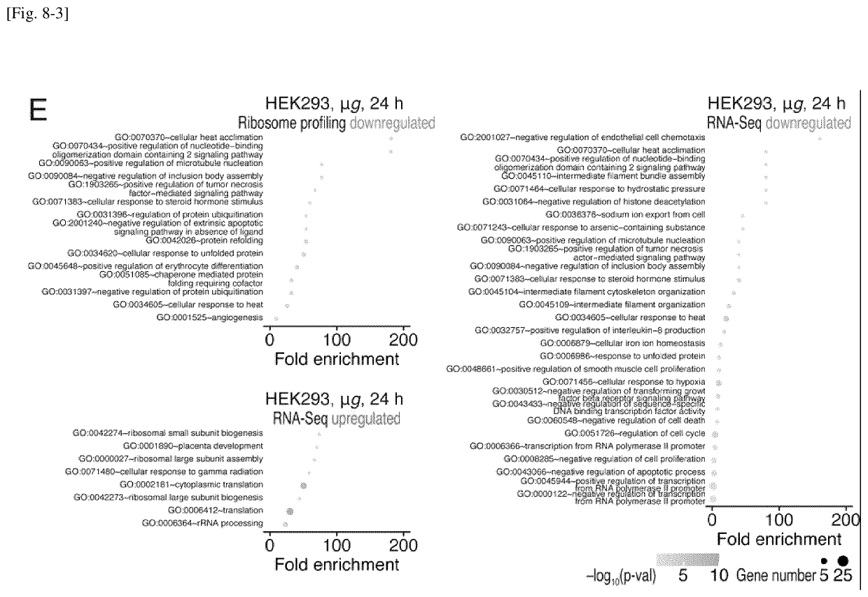

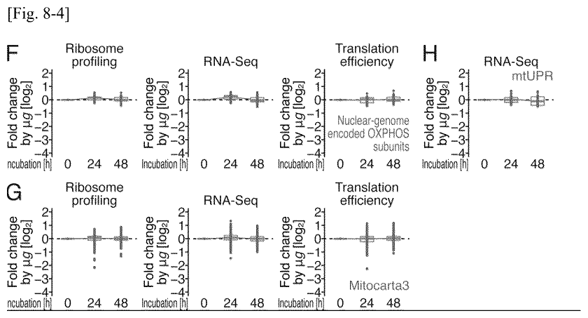

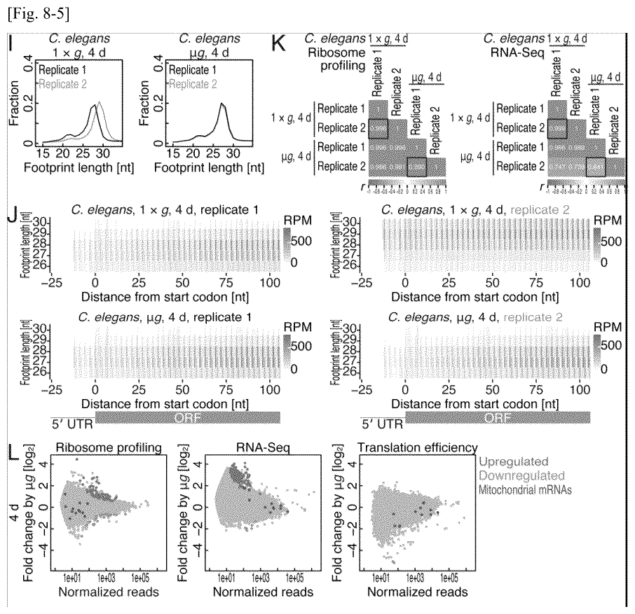

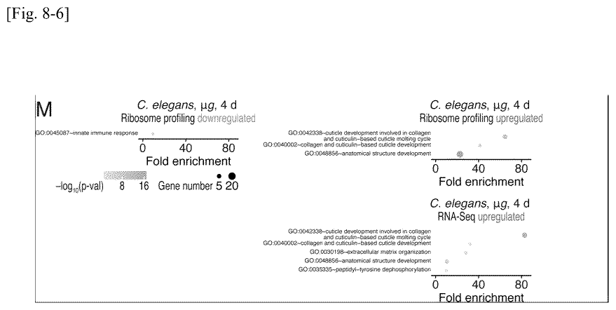

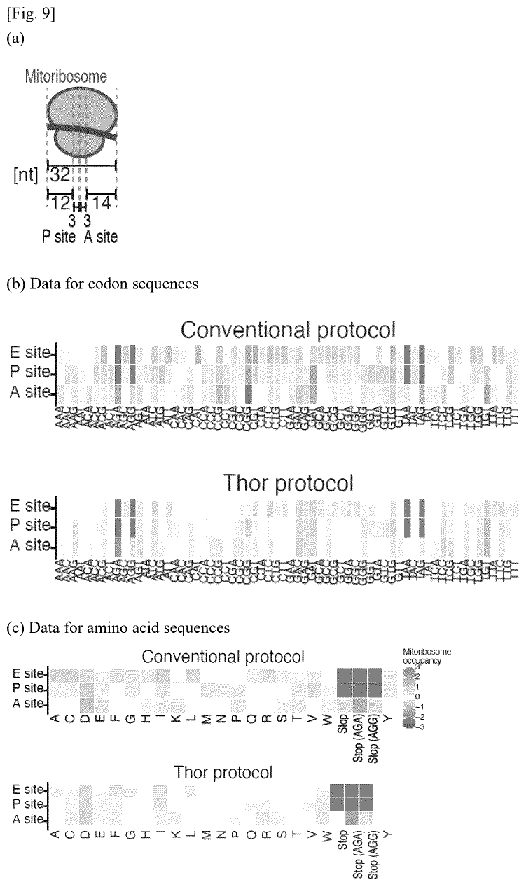

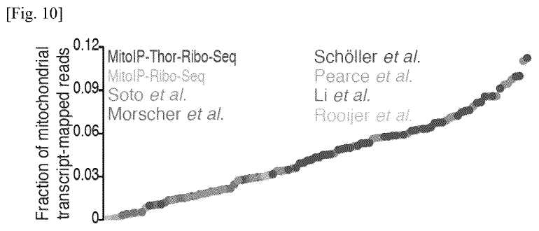

[FIG. 4-1] Preparation of library from low input by the Thor-Ribo-Seq method. (A) Schematic representation of the experimental design to verify the potential of Thor-Ribo-Seq. In the “digested-then-aliquoted” experiment, the lysate was treated with RNase I and then divided into samples of different concentrations. In the “aliquoted-then-digested” experiment, the lysate was first subjected to concentration adjustment and then digested with RNase I. The symbol “~” used in the drawing means about (approximately).

[FIG. 4-2] (B) Gel photograph of complementary RNA for the ribosome footprints amplified by in vitro transcription. (C) Gel photographs of PCR-amplified DNA library for which initial materials having adjusted concentrations were prepared by the Thor-Ribo-Seq method. The symbol “~” used in the drawing means about (approximately).

[FIG. 5-1] Results of experiments performed to investigate Thor-Ribo-Seq bias due to low input are shown. (A and E) Distribution of Thor-Ribo-Seq footprint lengths obtained under the “digested-then-aliquoted” condition (A) and the “aliquoted-then-digested” condition (E). (B to D and F to H) Metagene plots of the 5′ end positions of ribosome footprints around the start codon in the indicated experiments. The X-axis represents the position relative to the start codon (the first nucleotide of the start codon is 0); the Y-axis represents footprint length; and the color scale indicates read abundance. RPM: reads per million. The symbol “~” used in the drawings means about (approximately).

[FIG. 5-2] Results of experiments performed to investigate Thor-Ribo-Seq bias due to low input are shown. (A and E) Distribution of Thor-Ribo-Seq footprint lengths obtained under the “digested-then-aliquoted” condition (A) and the “aliquoted-then-digested” conditions (E). (B to D and F to H) Metagene plots of the 5′ end position of ribosome footprints around start codon in the indicated experiments. The X-axis represents the position relative to the start codon (the first nucleotide of the start codon is 0); the Y-axis represents footprint length; and the color scale indicates read abundance. RPM: reads per million. The symbol “~” used in the drawings means about (approximately).

[FIG. 5-3] (I) Pearson's correlation coefficients (r) among the indicated experiments. Read duplications generated by in vitro transcription were suppressed with the UMI in the first linker. The r-value scale is shown with color bars. (J) Gene numbers (3 reads or more) detected with 4 million footprint reads in the indicated experiments. Representative transcripts in each gene defined in the MANE Select (Ensembl) were used. (K) Correspondence of averaged ribosome occupancy on A-site codon sequences in Thor-Ribo-Seq (digested-then-aliquoted) with low material inputs. The symbol “~” used in the drawings means about (approximately).

[FIG. 5-4] (L) Schematic representation of the suppression of duplicated reads with UMI. (M) Pearson's correlation coefficients (r) among the experiments indicated. Duplicated reads generated by PCR were suppressed with UMIs in the first and second DNA linkers. The scale of the r-value is indicated with color bars. The symbol “~” in the drawings means about (approximately).

[FIG. 6-1] Outline of the steps of the DNA library creation scheme of the present invention (SEQ ID NOS: 16 to 28).

[FIG. 6-2] Outline of the steps of the DNA library creation scheme of the present invention (SEQ ID NOS: 16 to 28) (continued). NI799 is mentioned in the drawing as the third oligonucleotide, but NI822 to 832 disclosed in McGlincy, N. J. & Ingolia, N. T. 2017 (Non-patent document 7) can also be used.

FIG. 7 Genome-wide ribosome profiling revealed microgravity-induced mitochondrial translation repression in humans and nematode. (A and D) Schematic views of the experiments for HEK293 cells (A) and nematode (D) performed on the International Space Station. μg, microgravity. (B) MA (M, log ratio; A, average) plots of ribosome footprint change (left), RNA abundance change (middle), and translation efficiency change (right) in HEK293 cells cultured under microgravity for 24 hours. Significantly altered transcripts (false discovery rate [FDR]<0.05) and mitochondrial genome-encoded mRNAs are highlighted. (C) Box-and-whisker plots of ribosome footprint change (left), RNA abundance changes (middle), and translation efficiency changes (right) of mitochondrial genome-encoded mRNAs over time in microgravity culture. (E) Cumulative distributions of ribosome footprint change (left), RNA abundance change (middle), and translation efficiency change (right) of nuclear genome-encoded mRNA and mitochondrial genome-encoded mRNA of nematode during 4 days of microgravity culture. The p-values were calculated by Mann-Whitney U test.

[FIG. 8-1] Characteristics of ribosome profiling data in space flight samples. (A) Lengths of ribosome footprints of the samples indicated. (B) Metagene plots relative to the start codon across the footprint lengths of the ribosome footprints. The positions of the 5′ ends of the ribosome footprints are depicted. Colors indicate the amounts of reads. RPM, reads per million. (C) Pearson's correlation coefficients (r) among samples specified in ribosome profiling and RNA-Seq. Black squares indicate correlation of biological replicates.

[FIG. 8-2] Characteristics of ribosome profiling data in space flight samples (continued). (D) MA (M, log ratio; A, average) plots of ribosome footprint change (left), RNA abundance change (middle), and translation efficiency change (right) in HEK293 cells after 48 hours of culture under microgravity (μg). Significantly altered transcripts (false discovery rate [FDR]<0.05) and mRNAs encoded in the mitochondrial genome are highlighted.

[FIG. 8-3] Characteristics of ribosome profiling data in space flight samples (continued). (E) Gene Ontology (GO) analysis was performed for genes of which expressions were down- or up-regulated by microgravity. The colors indicate statistical significance, and the size of each circle indicates the number of genes found in that GO category. The p-values were calculated by a modified Fisher's exact test (EASE score).

[FIG. 8-4] Characteristics of ribosome profiling data in space flight samples (continued).

(F and G) Box-and-whisker plots of ribosome footprint change (left), RNA abundance change (center), translation efficiency change (right) of the nuclear genome-encoded OXPHOS subunits (F) and mitochondria-localized proteins (G) (defined by Mitocarta3) of HEK293 cell measured over time in microgravity culture of HEK293 cells. (H) Box-and-whisker plot of the RNA abundance change of mtUPR target genes (center) over time in microgravity culture of HEK293 cells.

[FIG. 8-5] Characteristics of ribosome profiling data in space flight samples (continued). (I) Lengths of ribosome footprints of the samples indicated. (J) Metagene plots relative to the start codon across the footprint lengths of the ribosome footprints. The positions of the 5′ ends of the ribosome footprints are depicted. Colors indicate the amounts of reads. RPM, reads per million. (K) Pearson's correlation coefficients (r) among samples specified in ribosome profiling and RNA-Seq. Black squares indicate correlation of biological replicates. (L) MA (M, log ratio; A, mean) plots of ribosome footprint change (left), RNA abundance change (middle), and translation efficiency change (right) in nematode during 4 days of microgravity culture. Significantly altered transcripts (false discovery rate [FDR]<0.05) and mRNAs encoded in the mitochondrial genome are highlighted.

[FIG. 8-6] Characteristics of ribosome profiling data in space flight samples (continued). (M) Gene Ontology (GO) analysis was performed for genes of which expressions were down- or up-regulated by microgravity. Colors indicate statistical significance, and the size of each circle indicates the number of genes found in that GO category. The p-values were calculated by a modified Fisher's exact test (EASE score).

[FIG. 9] (a) Schematic representation of the relative positions of P and A sites on the footprint of the mitochondrial ribosome (mitoribosome). (b) and (c) The occupancies of mitoribosomes at A, P, and E sites observed in MitoIP-Ribo-Seq (top) and MitoIP-Thor-Ribo-Seq (bottom) are shown. The data for codon sequence (b) and amino acid sequence (c) are shown. The color scale indicates mitoribosome occupancy.

FIG. 10 Comparison of the percentages of mapped reads of mitochondrial transcripts found in MitoIP-Thor-Ribo-Seq and the published conventional MitoRibo-Seq. The published data are from Rooijers et al. (Reference 69), Pearce et al. (Reference 70), Morscher et al. (Reference 71), Li et al. (Reference 72), Scholler et al. (Reference 73), and Soto et al. (Reference 74).

### MODES FOR CARRYING OUT THE INVENTION

The following explanations of the present invention may be made with reference to representative embodiments or specific examples, but the present invention is not limited to such embodiments or specific examples. In this description, a numerical value range expressed by using “to” means a range that includes the numerical values mentioned before and after “to” as the lower and upper limits. In this description, when a numerical value is accompanied by the term “about”, it is intended to mean a range including ±10% of that value. The symbol “nt” used for RNA length means nucleotide.

Next-generation sequencers can simultaneously process tens to hundreds of millions of randomly truncated DNA fragments in parallel, yielding data ranging from 1 billion bases (1 gigabase) to 1 trillion bases (1 terabase) in a single sequencing run. In addition to whole genome sequencing, next-generation sequencers are used in studies such as targeted sequencing, which targets genomic regions relating to specific research targets such as diseases, and epigenetics such as methylation sequencing. Next-generation sequencers are also used to study the central dogma of molecular biology, namely the concept that genetic information is transmitted in the order of “DNA→(transcription)→mRNA (messenger RNA)→(translation)→protein”. For example, RNA-Seq (RNA sequencing) uses a next-generation sequencer to reveal the presence and amount of RNA in a biological sample at a specific moment in time and enables comprehensive gene expression analysis. Furthermore, in research to elucidate how the reaction of translation, in which proteins are produced from mRNA, is regulated, ribosome profiling, a technique using next-generation sequencers, can be used to comprehensively analyze what codons of what mRNA are decoded by the ribosomes, providing a bird's-eye view of the state of translation.

RNA-seq (RNA sequencing) is a technique that enables genome-wide profiling at gene expression level. In this description, RNA-seq includes the whole transcriptome sequencing (total RNA-seq), which provides a comprehensive picture of the transcription profile of a cell at a biological moment, as well as target RNA sequencing, which measures only target transcripts to analyze expression differences and allele-specific gene expression, transcriptional and translational regulation-related small noncoding RNA (e.g., transfer RNA, snoRNA, snRNA etc.) sequencing, and microRNA sequencing. snRNA (small nuclear RNA) is a class of small RNAs that exist in the nuclei of eukaryotic organisms and are involved in various reaction processes, including RNA splicing and rRNA processing, along with other proteins. snoRNA (small nucleolar RNA) is a class of small RNAs involved in chemical modifications (such as methylation and pseudouridylation) of rRNA and other RNAs. MicroRNA, classified as functional noncoding RNA, is a class of functional nucleic acids that are encoded on the genomes and undergo a multistep production process to finally become microRNAs of 20 to 25 bases in length, which are involved in the regulation of basic life processes such as cell development, differentiation, proliferation and cell death.

The term transcriptome refers to total transcripts (total RNA) in a cell. The term translatome refers to the totality of RNAs used for translation in a cell at a given biological moment.

Ribosome profiling (Ribo-Seq) uses the fact that when an mRNA molecule is degraded by an enzyme or the like, a part of the mRNA to which the ribosome was bound is protected from degradation and remains to determine the sequences of a number of portions of mRNAs to which the ribosomes were bound and to determine the region of mRNA that was actively translated in the cell at a particular time.

The term “ribosome footprint” or “footprint” used in this description refers to a portion of mRNA that has been protected from degradation by enzymes or the like in ribosome profiling and remains. The length of the footprint is about 40 nt or less, generally about 30 nt.

The term “read” or “sequence read” used in this description generally means a data sequence of A, T, C, and G bases determined for a DNA or RNA sample. The read or sequence read referred to in this description is, in particular, a sequence determined for cDNA produced by the method of the present invention.

The term “non-ribosomal RNA” used in this description is used as a generic term for RNA other than ribosomal RNA (rRNA). Examples of non-ribosomal RNA include mRNA, transfer RNA (tRNA), mitochondria-derived transfer RNA (Mt-(RNA), chloroplast-derived transfer RNA, snRNA, snoRNA, microRNA, and so forth.

In this description, the term “non-coding RNA” is used as a generic term for RNA that does not encode protein, and examples of non-coding RNA include IRNA, transfer RNA (tRNA), mitochondria-derived ribosomal RNA (Mt-rRNA), mitochondria-derived transfer RNA (Mt-tRNA), chloroplast-derived ribosomal RNA, chloroplast-derived transfer RNA, snRNA, snoRNA, microRNA, and so forth.

The term “sample containing RNA” used in this description means a crude extract of cell or tissue obtained by lysing or disrupting single cell, cell population, cultured cells or tissue containing at least non-ribosomal RNA (henceforth referred to as lysate), and the single cell, cell population, cultured cells or tissue can be derived from any organism. Specifically, examples of the lysate include lysates of bacteria, fungi, animal cells or tissues, plant cells or tissues, and cultured cells thereof, but are not limited to these. The lysate can be prepared by cell lysis using a surfactant or physical disruption (e.g., mechanical disruption, homogenization in solution, sonication, freeze-thawing, disruption with mortar and pestle, etc.), and the preparation method can be appropriately selected according to the species of organism or type of cell or tissue.

The term “RNA molecule” or “RNA molecule of interest” used in this description refers to any RNA molecule in a sample containing RNA, for example, any RNA molecule that can be an object of sequencing and/or quantification analysis in a DNA library prepared by the method of the present invention. The RNA molecule may be mRNA, transfer RNA (tRNA), mitochondria-derived transfer RNA (Mt-tRNA), chloroplast-derived transfer RNA, snRNA, snoRNA, microRNA, or the like.

The term “DNA library” used in this description can be a DNA library containing cDNA constructed from RNA. cDNA is generally complementary DNA and often refers to a DNA fragment obtained by reverse transcription of RNA, but in this description, it also refers to a DNA fragment obtained by the method of the present invention (i.e., in vitro transcription, reverse transcription, and in some cases PCR). The DNA library includes, for example, and not limited to, RNA expression library and ribosome footprint library. cDNA in a DNA library can contain one or more additional DNA sequences.

The term “linker” used in this description refers to an oligonucleotide that is linked to an RNA molecule by ligation. In a specific embodiment, the linker can contain a promoter recognized by a polymerase, a sequence that provides a sequencing primer-binding site, a flow cell primer and/or a barcode (nucleotides constituting unique molecular identifier or sample identifier). In addition, the linker can contain a 3′-end modification to prevent self-ligation. The 5′ end of the linker can be phosphorylated so that it can be App-modified (adenylated) by Mth ligase. If the promoter sequence recognized by a polymerase is the T7 promoter, it can be adjusted so that the end is GGG, and an optimization sequence (e.g., a sequence to promote transcription) can optionally be introduced into upstream or downstream sequence thereof. The linker can be about 20 to 200 nt, about 25 to 150 nt, or about 30 to 100 nt, in length.

The terms “first oligonucleotide”, “second oligonucleotide”, and “third oligonucleotide” (hereinafter also collectively referred to as “first, second, and third oligonucleotides”) used in this description refer to oligonucleotides designed so that they can hybridize to at least a part of the linker and/or template DNA, especially, oligonucleotides that function as substrates of polymerization of nucleotides by polymerase. The first, second and third oligonucleotides can be about 20 to 200 nt, about 25 to 150 nt, or about 30 to 100 nt, in length. The first oligonucleotide can be designed so as to be complementary to a promoter sequence recognized by a polymerase, such as the T7 promoter sequence, and optimization sequences upstream and downstream thereof, and to have a Tm value higher than 37° C. As the second and third oligonucleotides, those described in McGlincy, N. J. & Ingolia, N. T. 2017 (Non-patent document 7) can be used (e.g., NI-798 and NI-799). The second oligonucleotide can be a primer for reverse transcription with phosphorylated 5′ end. The second oligonucleotide can further be a forward primer for PCR used in making a single-stranded DNA (ssDNA) into double-stranded DNA (dsDNA). The third oligonucleotide is a reverse primer for PCR. The second and third oligonucleotides may be flow cell primers necessary for next generation sequencing analysis.

The term “sequencing primer” used in this description refers to a primer for sequencing. The sequencing primer is annealed to a template strand during the cluster formation stage. The cluster formation is performed to increase the amount of DNA to a sufficient amount that is detectable during sequencing. The cluster is an amplified region of DNA on the surface of a flow cell that is to be sequenced. Sequencing begins with the extension of an initial sequencing primer, generating the first read with each cycle. When fluorescently labeled nucleotides are added in competition to a growing chain, only one is incorporated at a time based on the template sequence. The cluster is excited by a light source and emits a characteristic fluorescence signal. This process is called sequencing by synthesis, where the number of cycles determines the lengths of the read and emission wavelength also varies depending on the incorporated nucleotide. In FIG. 6, they are described as Read #1 Primer and Read #2 Primer. These sequences are essential for next-generation sequencing analysis (e.g., analysis with a next-generation sequencing of Illumina, Inc).

The term “flow cell primer” used in this description refers to a sequence that is responsible for binding a DNA library to flow cells. This sequence is essential for next-generation sequencing analysis (e.g., analysis using next-generation sequencer of Illumina, Inc). The flow cells have oligonucleotides growing like lawn, which are complementary to the flow cell primers, where the single stranded cDNAs (DNA fragments) of the DNA library hybridize via the flow cell primer portions. In FIG. 6, they are indicated as Flow cell Primer #1 and Flow cell Primer #2.

The term “unique molecular identifier” or “UMI” used in this description refers to a random, non-random, or semi-random molecular tag that can be added to an RNA molecule. If it is added to an RNA molecule with a linker and the molecule is incorporated into a DNA library, the UMI can be used to correct amplification bias to be introduced during subsequent PCR steps by identifying the sequence of the unique molecular identifier (UMI) sequenced after the amplification, and counting reads having the same sequences of the combination UMI-RNA (combination of UMI and RNA molecule of interest) only once. The UMI can be created, for example, during nucleic acid synthesis by performing the synthesis with the four base species in a ratio of 1:1:1:1, and at this stage, the sequence is randomized. The length of UMI may be, for example, 4 to the power of 5, about 1000, and therefore the probability of a coincidental match thereof is about 1/1000, which is almost negligible. UMIs may exist contiguously or separately (e.g., in the examples described below, they are linked on the 5′ and 3′ sides of the RNA molecule of interest). The total length of the UMIs can be 5 to 10 nt.

The term “sample identifier” used in this description refers to a molecular tag that can be added to an RNA molecule and that identifies the origin of the RNA molecule of interest. That is, it is a molecular tag for extracting the sample from which the RNA molecule of interest is derived. Each sample is tagged with a different sample identifier sequence, e.g., one sequence is added to each sample and different sequences are added for different samples to pool the tagged samples. The pooled samples are sequenced and the sample identifier sequence can be used to determine the origin of the sequences. In the present invention, as the sample identifier, the sequences disclosed in Sasagawa et al. (2018) (Reference 55) can be used.

In this description, the term “barcode” refers to a molecular barcode, specifically, either or both of a unique molecular identifier and a sample identifier. In some embodiments, cDNAs in a DNA library can contain barcodes (unique molecular identifiers or sample identifiers) to aid downstream processes such as error correction, identification, or sequencing.

The present invention relates to a method for preparing a DNA library from a sample containing RNA, comprising
(1) the step of ligating a first DNA linker to the 3′ end of at least one RNA molecule in a sample containing RNA to obtain an RNA molecule having the first DNA linker, wherein the first DNA linker comprises at least a promoter antisense sequence;
(2) the step of hybridizing a first oligonucleotide with the RNA molecule having the first DNA linker to obtain the RNA molecule having a double-stranded DNA promoter region, wherein the first oligonucleotide comprises a promoter sense sequence;
(3) the step of obtaining a complementary strand of the RNA molecule having the first DNA linker (complementary RNA) by in vitro transcription using the RNA molecule having the double-stranded DNA promoter region as a template and an RNA polymerase that recognizes the promoter sequence and initiates transcription; and
(4) the step of ligating a second DNA linker to the 3′ end of the complementary RNA and further obtaining single-stranded DNA by reverse transcription using the complementary RNA having the second DNA linker as a template.

The above method can further comprise the following step (5):
(5) the step of obtaining a double-stranded DNA by PCR using the single-stranded DNA obtained in the step (4) as a template.

Further, the present invention also relates to a method for sequencing and/or quantifying an RNA molecule in a sample containing RNA, comprising
the step of performing the steps (1) to (4) described above and optionally further performing the step (5) described above to prepare a DNA library; and
the step of analyzing the DNA library by a next-generation sequencing method to obtain data of sequencing and/or quantification of the RNA molecule in the sample containing RNA.

According to the method of the present invention for preparing a DNA library from a sample containing RNA, the DNA library is prepared as follows: an RNA molecule having a first DNA linker is obtained by ligating the first DNA linker to the 3′ end of an RNA molecule contained in the sample containing RNA, then a first oligonucleotide is hybridized with the RNA molecule having the first DNA linker to obtain the RNA molecule having a double-stranded DNA promoter region, a complementary strand of the RNA molecule having the first DNA linker (referred to as complementary RNA in this description) by in vitro transcription using the RNA molecule having a double-stranded DNA promoter region as a template, a second DNA linker is further ligated to the 3′ end of the complementary RNA, and this complementary RNA is used as a template (RNA-DNA chimera template) to perform reverse transcription reaction and thereby obtain a single-stranded DNA to create the library (FIG. 6). That is, in the method of the present invention, nucleic acid replication reactions are performed by using an RNA-DNA chimera molecule as a template in at least two steps: i) the in vitro transcription using the RNA molecule having the first DNA linker as a template and an RNA polymerase that recognizes a promoter sequence to initiate transcription, and ii) the reverse transcription using the complementary RNA having the second DNA linker as a template. According to the present invention, for the in vitro transcription of i), an RNA polymerase derived from phage and showing a fast transcription rate is used to amplify RNA, thereby RNA is efficiently amplified, and therefore a DNA library can be created from a small amount of RNA material. As the RNA polymerase that can be used for the in vitro transcription of i), any RNA-dependent RNA polymerase that can recognize the promoter sequence to initiate transcription and efficiently synthesize RNA can be used.

The first DNA linker is a single stranded DNA (ssDNA) and can contain at least a promoter antisense sequence. The first oligonucleotide is ssDNA and can contain at least a promoter sense sequence. In one embodiment of the present invention, the RNA polymerase used in the step of (3) can be a phage-derived RNA-dependent RNA polymerase. The first DNA linker ligated to the 3′ end of the RNA molecule and the first oligonucleotide are complementary to each other and can hybridize with each other, whereby the RNA molecule can have a double-stranded promoter region. The RNA-dependent RNA polymerase used for the in vitro transcription (step of (3)) of the present invention recognizes that double-stranded DNA promoter region and initiates transcription, and thereby a complementary strand of the RNA molecule ligated with the first DNA linker at the 3′ end can be synthesized. As the promoter incorporated into the first DNA linker and the first oligonucleotide, a promoter specific to the RNA-dependent RNA polymerase employed can be selected.

The second DNA linker is ssDNA, and comprises a sequence that provides a sequencing primer (e.g., Read #1 Primer mentioned in FIG. 6)-binding site to cDNAs of the DNA library.

In a specific embodiment of the present invention, the RNA polymerase used in the step (3) can be T7 RNA polymerase. RNA amplification by in vitro transcription using T7 RNA polymerase is a useful option for handling small amounts of nucleic acids (References 14 and 15, a list of references is provided at the end of this description) and has been reported to be a less biased approach than exponential expansion by PCR because it is linear amplification (Reference 16). In fact, linear amplification has been introduced into a variety of single-cell sequencing techniques, including whole genome analysis (Reference 17), RNA-Seq (References 18 and 19), epigenomic profiling (References 20 and 21), etc. Similarly, the investigation of the limited transcriptomes on RNA-binding proteins has been greatly improved by this approach (References 22 and 23). However, in these methods, dsDNA is used as the template for the in vitro transcription using T7 RNA polymerase, and the methods could only be performed at a later stage of the library creation. In contrast, in the method of the present invention, the in vitro transcription using T7 RNA polymerase is performed with an RNA template, and RNA is amplified at an early stage in the procedure. Amplification of RNA prior to dsDNA synthesis is beneficial for sequencing techniques targeting RNA. This is because performing the amplification at an early stage in the procedure effectively reduces loss of RNA material.

T7 RNA polymerase can synthesize a complementary RNA from an RNA template bound to the dsDNA T7 promoter region (Reference 25). The method of the present invention for preparing a DNA library from a sample containing RNA can be designed so that the first DNA linker and the first oligonucleotide form the dsDNA T7 promoter sequence (sense strand 5′-TAATACGACTCACTATAGGG-3′ (SEQ ID NO: 6), antisense strand 5′-CCCTATAGTGAGTCGTATTA-3′ (SEQ ID NO: 7)).

In a specific embodiment of the present invention, a flanking region of the T7 promoter sequence can contain a sequence that increases the transcription efficiency. As an example of the sequence that increases the transcription efficiency, following GGG on the 3′ side of the T7 promoter sense sequence, an AT-rich sequence, for example, “5′-ATAAT-3” (Reference 26) or “5′-AAATA-3” mentioned in Reference 26, can be added. The sequence “ATAAT”, which increases the transcription efficiency, provides about 5-fold higher transcription activity than the motif “5′-TTCCC-3” compared in Reference 26. Further, an AT-rich motif such as “5′-GAATT-3” or “5′-AAATA-3′” mentioned in Reference 26 can be added immediately upstream of the 5′ end of the T7 promoter sense sequence, thereby increasing the activity of the T7 promoter at low template concentration (Reference 26). In one embodiment of the present invention, the first DNA linker and the first oligonucleotide contain a sequence that increases the transcription efficiency in a flanking region of the promoter sequence. In a specific embodiment of the present invention, the T7 promoter sense sequence containing a sequence that increases the transcription efficiency is 5′-GAATTTAATACGACTCACTATAGGGATAAT-3′ (SEQ ID NO: 8) and the T7 promoter antisense sequence is 5′-ATTATCCCTATAGTGAGTCGTATTAAATTC-3′ (SEQ ID NO: 9).

In the present invention, the method for preparing a DNA library from a sample containing RNA can include, as a pretreatment, the step of lysing or crushing cells to obtain a lysate. The lysate should preferably be prepared with a gentle means, e.g., cell lysis using a surfactant, to avoid degradation or damage of ribosomes. For the same reason, the lysate should preferably be prepared without any protein denaturing agent, Mg²⁺ chelating agent, or organic solvent such as phenol or chloroform. Since DNA inhibits the subsequent DNA synthesis, it may be degraded by using, for example, DNase. Furthermore, the lysate can also be obtained in the presence of a protein translation inhibitor, cycloheximide. In one embodiment, the lysate can be obtained as a supernatant obtained by suspending cells in a buffer containing a surfactant and cycloheximide, incubating them in the presence of DNase I (RNase-free), and centrifuging the cell suspension, as described in the in the examples mentioned below.

For the preparation of a DNA library for organelle translation analysis, organelles, e.g., mitochondria and chloroplasts can be isolated from the cell lysate and purified. Organelles can be concentrated by sucrose density gradient centrifugation using ultracentrifugation or by immunoprecipitation using antibodies that bind to membrane proteins present on the organelle outer membranes. In one embodiment of the present invention, mitochondria can be concentrated by using an immunoprecipitation method comprising incubating a cell lysate with beads conjugated with anti-TOM22 antibody.

The method of the present invention can be used to create a DNA library from a small amount of RNA material. The small amount of RNA material can be, for example, but is not limited to, RNA from rare and valuable samples or samples that are difficult to prepare. In one embodiment of the present invention, the sample containing RNA can be a lysate of less than 1×10⁶ cells. In a specific embodiment, the sample containing RNA can be a lysate of 5×10⁵ cells or less, 1×10⁵ cells or less, 5×10⁴ cells or less, 1×10⁴ cells or less, 5×10³ cells or less, or 1×10⁸ cells or less. In a specific embodiment, the sample containing RNA contains 10 μg or less, 5 μg or less, 1 μg or less, 0.5 μg or less, 0.1 μg or less, 0.05 μg or less, or 0.01 μg or less of RNA.

In a specific embodiment of the present invention, the at least one RNA molecule in the sample containing RNA is mRNA or a fragment thereof, in particular, a ribosome footprint. Since RNA molecule, in particular, ribosome footprint, may have a 3′-end phosphate due to an enzymatic treatment, the 3′ end can be dephosphorylated with a 3′ phosphatase (e.g., T4 polynucleotide kinase) prior to ligating the first DNA linker, if necessary.

Ligation to the 3′ hydroxy end of the RNA molecule requires a 5′ adenylated linker. 5′ Adenylation of the ssDNA linker may be performed with Mth RNA ligase (5′ DNA Adenylation Kit, New England Biolabs) or 5′ adenylation modification may be introduced during DNA synthesis. Mth RNA ligase cannot adenylate the 5′ phosphate of ssDNA, thus reducing undesirable ligation products (such as concatemers and circular DNA). The preadenylated 5′ end of the ssDNA linker can be ligated to the 3′ end of the RNA molecule with T4 RNA ligase 2, truncated KQ (New England Biolabs). However, the ligation of the ssDNA linker to the RNA molecule can be performed by any method that can efficiently achieve the ligation, other than the method using T4 RNA ligase. The 3′ end of the ssDNA linker can be modified with, for example, 2′,3′-dideoxycytidine, C3 spacer, biotinylation, etc. to prevent ligation reactions of the linkers themselves.

Following the above ligation reaction, unreacted linkers can be subjected to lambda-nuclease digestion. If the linker is adenylated, deadenylase treatment should be performed prior to the lambda-nuclease digestion.

Each of the steps of the method for preparing a DNA library from a sample containing RNA will be explained below.

In one embodiment of the present invention, the ligation reaction of the step (1) can be is carried out by incubating an RNA molecule (RNA molecule of which 3′ end has been dephosphorylated, if necessary) and a pre-adenylated first DNA linker with T4 RNA Ligase 2, truncated KQ (New England Biolabs) in a reaction solution (17.5% PEG8000, T4 RNA Ligase Reaction Buffer) at, for example, 22° C. for 3 hours to overnight. Other than T4 RNA Ligase 2, truncated KQ, Rnl2, Rnl2 truncated, and Rnl1 can be used (provided that adenylated linker cannot be used with these).

The step (2) is a step of hybridizing the first oligonucleotide to the RNA molecule having the first DNA linker to obtain the RNA molecule having a double-stranded DNA promoter region. In one embodiment of the present invention, the step (2) can be performed by incubating the RNA molecule having the first DNA linker and the first oligonucleotide in a reaction solution used for in vitro transcription.

In one embodiment of the present invention, the in vitro transcription of the step (3) is performed by contacting the RNA molecule having a double-stranded DNA promoter region as a template with a reaction solution containing substrate NTPs (final concentration 10 to 200 mM), magnesium ion (final concentration 1 to 30 mM), a reducing agent such as DTT (final concentration 10 to 200 mM), and RNase-free water, for example, at 37° C. for about 10 minutes to 5 hours. The reaction solution can contain a polyamine such as spermidine (final concentration 1 to 2 mM) and the nonionic surfactant Triton X-100 (final concentration 0.001 to 0.01%). The in vitro transcription can be performed by using a kit such as T7-Scribe Standard RNA IVT Kit (CELLSCRIPT), ScriptMAX (registered trademark) Thermo T7 Transcription Kit (TOYOBO) and T7 RiboMAX (registered trademark) Express Large Scale RNA Production System. Those skilled in the art can appropriately optimize the temperature and time and the composition of the reaction solution for the in vitro transcription according to the types of the enzyme and template.

In one embodiment of the present invention, the ligation reaction of the second DNA linker of the step (4) can be performed by incubating the complementary RNA amplified in the step (3) and the pre-adenylated second DNA linker with T4 RNA Ligase 2, truncated KQ (New England Biolabs) in a reaction solution (17.5% PEG8000, T4 RNA Ligase Reaction Buffer), for example, at 22° C. for 3 hours to overnight. Other than T4 RNA Ligase 2, truncated KQ, Rnl2, Rnl2 truncated, Rnl1 can be used (provided that adenylated linker cannot be used with these).

The reverse transcription of the step (4) is a technique well known in the art and can be performed in the present invention by methods commonly practiced in the art. For example, it can be performed in a reaction solution containing a reverse transcriptase (e.g., ProtoScript II Reverse Transcriptase (1 U/μL)), complementary RNA with a second DNA linker as a template, second oligonucleotide (final concentration 0.125 μM), substrate NTPs (final concentration 5 mM), buffer, DTT (final concentration 5 mM), RNase inhibitor, and RNase-free water. The reverse transcription reaction usually comprises three steps: annealing of primer (65° C., 1 minute, =>4° C., >1 minute), cDNA synthesis (37 to 50° C., 10 to 90 minutes), and inactivation of enzyme (70 to 85° C., 5 to 15 minutes). Those skilled in the art can appropriately optimize the temperature and time and the composition of the reaction solution for the reverse transcription reaction according to the types of enzyme and template. The reverse transcription can be performed by using ProtoScript II Reverse Transcriptase (New England Biolabs), SuperScript (registered trademark) II Reverse Transcriptase (Thermo Fisher Scientific), SuperScript (registered trademark) III Reverse Transcriptase (Thermo Fisher Scientific), SuperScript (registered trademark) IV Reverse Transcriptase, etc. A single-stranded DNA (ssDNA) is obtained by the reverse transcription of the step (4).

In one embodiment of the present invention, the reverse transcription of the step (4) can be performed by using a sequence-specific primer, e.g., the second oligonucleotide. The second oligonucleotide is ssDNA, which can hybridize to the second DNA linker ligated to the complementary RNA molecule and is set to be of a sufficient length in terms of Tm value. In one embodiment of the present invention, the primer for the reverse transcription in the step (4) is not oligo (dT) primer.

In one embodiment of the present invention, a double-stranded DNA can be obtained by using ssDNA as a template in PCR of the step (5) of the method for preparing a DNA library. PCR is a technique well known in the art and can be performed also in the present invention by methods commonly used in the art. For example, it can be performed in a reaction solution containing a DNA polymerase (e.g. Phusion polymerase (2 U/μL)), ssDNA as a template, a pair of primer oligonucleotides (final concentration 0.5 to 1 μM), substrate NTPs (final concentrations 1 to 100 mM), buffer, DTT and water. PCR typically comprises 20 to 40 cycles of three steps, denaturation (94 to 98° C.), annealing (55 to 70° C.), and extension (68 to 72° C.) (5 to 30 seconds for each step). It can also be performed with two steps, denaturation and annealing/extension. The initial denaturation step (94 to 98° C.) can be performed for 1 to 3 minutes. The final extension step can be performed for 10 seconds to 5 minutes. Those skilled in the art can appropriately optimize the temperature and time of each step and the composition of the reaction solution for PCR according to the types of enzyme and template. PCR can be performed by using Phusion High-Fidelity DNA Polymerase, KAPA TAQ DNA polymerase, etc.

The DNA library prepared by the method of the present invention can be a library containing single-stranded DNA (ssDNA) or double-stranded DNA (dsDNA), preferably a library containing dsDNA. The DNA library prepared by the method of the present invention can be a library containing cyclic DNA or linear DNA, but is preferably a library containing linear DNA, more suitably linear dsDNA. In a specific embodiment, the DNA library created by the method of the present invention contains neither cyclic dsDNA nor cyclic ssDNA.

In one embodiment of the present invention, the pair of primer oligonucleotides used in PCR can be designed to anneal to the 3′ end regions of the sense and antisense strands of the ssDNA obtained by the reverse transcription, respectively. As one of the pair of primer oligonucleotides used in the reaction of PCR, one having the same sequence as that of the second oligonucleotide used in the reverse transcription may be used, or an oligonucleotide having a different sequence may be used. Among the pair of primer oligonucleotides used in the reaction of PCR, the reverse primer that anneals to the sense strand of the ssDNA obtained by the reverse transcription may be referred to as third oligonucleotide in this description. In the case of preparing a next-generation sequencing cDNA library, the pair of primer oligonucleotides used in PCR comprise a flow cell primer for nucleic acid immobilization.

In one embodiment of the present invention, the DNA library can be a next-generation sequencing cDNA library.

In the present invention, a method using a next-generation sequencer can be employed as a sequencing technique for sequencing. The type of the next-generation sequencer is not particularly limited, but examples include HiSeq2000 (Illumina), Genome Analyzer IIx (Illumina), Genome Sequencer-FLX (Roche), etc. RNA sequencing using next-generation sequencers includes the step of immobilizing nucleic acids on flow cells or on microarrays. In the sequencing process, bridge amplification (especially bridge PCR) can occur in the flow cells with nucleic acids immobilized thereon or in the microarrays with nucleic acids immobilized therein.

The RNA sequencing is accomplished by using the “sequencing by synthesis (SBS)” technique. The SBS technique mentioned here refers to a technique for sequencing a nucleic acid by synthesizing the complementary strand of the target nucleic acid. The SBS technique may be selected from the group consisting of “pyrosequencing”, “sequencing by ligation”, and “sequencing by extension”. The “pyrosequencing” refers to a method of sequencing by detecting pyrophosphate produced upon nucleotide incorporation. The “sequencing by ligation” refers to a method of sequencing a nucleic acid by using a ligase for determining a nucleotide locating at a specified position within the nucleic acid sequence. The “sequencing by extension” refers to a nucleic acid sequencing method in which primers are extended with known or detectable nucleotides.

As the sequencing technique to perform the sequencing, “deep sequencing” can be employed. The “deep sequencing” refers to a method of sequencing multiple nucleic acids in parallel (Bentley et al. Nature 2008, 456:53-59). In a typical sequencing protocol using “deep sequencing”, nucleic acids (e.g., DNA fragments) are attached to the surface of a reaction platform (e.g., flow cells or microarrays). The attached nucleic acids are amplified in situ and can be used as a template for synthetic sequencing (e.g., SBS) using a detectable label (e.g., fluorescent reversible terminator deoxyribonucleotide). Typical reversible terminator deoxyribonucleotides can include 3′-O-azidomethyl-2′-deoxynucleoside triphosphates of adenine, cytosine, guanine, and thymine, each of which may be further labeled with mutually recognizable and removable fluorophores via linkers. The sequencing can also be performed by a single read method or paired-end method.

In one embodiment of the present invention, the method for preparing a DNA library from a sample containing RNA is performed to create a cDNA library for ribosome profiling. In this embodiment, the sample containing RNA can contain ribosome footprints. The RNA molecules in the sample containing RNA can be about 30 nt to about 40 nt. In one embodiment, the method for preparing a DNA library from a sample containing RNA of the present invention can be applied even if the RNA molecule of interest does not have any poly (A) tail. In a specific embodiment, the method for preparing a DNA library from a sample containing RNA of the present invention can also be applied when the RNA molecule of interest has a poly (A) tail. In a specific embodiment, when the RNA molecule of interest has a poly (A) tail, the step of degrading the poly(A) tail can be applied in advance in the method for preparing a DNA library from a sample containing RNA of the present invention.

Ribosome profiling can usually be performed on ribosome footprints obtained by a method comprising degrading RNA in a sample, such as lysate, and then removing ribosomal RNA (rRNA). Degradation of RNA can be performed by, for example, enzymatic degradation. RNA degradation refers to modifying RNA so that it becomes shorter in length than it was before the degradation, and the enzymatic degradation of RNA refers to degrading RNA by using an enzyme that can modify RNA so that it becomes shorter in length than it was before the degradation. The enzyme used may be a ribonuclease (RNA-degrading enzyme) such as endoribonuclease or exoribonuclease, and a single strand-specific RNA endonuclease, for example, RNase I can be used. Other examples of enzymes that can be used for the RNA degradation include RNase A, RNase TI, etc.

The RNA degradation can also be performed by partial alkaline hydrolysis. Partial alkaline hydrolysis can be performed by, for example, adding 10 μL of 2× alkaline hydrolysis solution (2 mM EDTA, 12 mM Na₂CO₃, 88 mM NaHCO₃, pH 9.3) to an equal volume of an RNA-containing solution (e.g. lysate), mixing them, incubating the mixture at 95° C. for 20 minutes, then placing the mixture on ice, and adding 300 μL of 0.3 M NaOAc (pH 5.2).

RNA molecules in the sample are degraded by the enzymatic degradation or partial alkaline hydrolysis, but a part of mRNA to which the ribosome is bound is protected from the degradation. A part of mRNA to which one ribosome is bound is called monosome. Monosomes in a sample containing mRNA and ribosome can be concentrated by sucrose density gradient centrifugation, sucrose cushion centrifugation, gel filtration chromatography using a spin column, or the like.

Removal of rRNA can be performed by the method using rRNA-subtraction oligonucleotides that can hybridize to rRNA and trap them on magnetic beads (Non-patent document 7, and Reference 29 in the list of references mentioned at the end of the section of Examples), or by using Ribo-Zero (registered trademark) rRNA Removal Kit (Illumina). The rRNA removal can also be performed by detaching the ribosome subunits from mRNA and removing the detached ribosome subunits, as described in WO2021/106814. The detachment of ribosome subunits from mRNA can also be carried out by any method, e.g., by removing Mg²⁺ ions necessary to maintain association of the both subunits. Mg²⁺ ions can be removed by any method, e.g., by using a chelating agent. That is, the step (a) of detaching the ribosome subunit from mRNA can be performed by using a chelating agent. Examples of the chelating agent include, for example, ethylenediaminetetraacetic acid (EDTA), nitrilotriacetic acid (NTA), diethylenetriaminepentaacetic acid (DTPA), glycol ether diamine-tetraacetic acid (EGTA, GEDTA), etc., and ethylenediaminetetraacetic acid (EDTA) is especially preferred. The concentration of the chelating agent can be 0.1 to 30 mM, preferably 5 to 15 mM, in the case of ethylenediaminetetraacetic acid (EDTA). The treatment with the chelating agent can be performed by placing the sample on ice for 30 seconds to 60 minutes in the case of EDTA, and the treatment time can be changed as needed. The removal of ribosomal subunits can be performed by employing any method that can remove the subunits on the basis of the sizes of the large and small subunits of ribosome, for example, ultrafiltration, size exclusion chromatography (SEC), etc.

According to one embodiment of the present invention, in the method for creating a DNA library for ribosome profiling, rRNA removal can be applied to the RNA molecule ligated with the first DNA linker obtained in the step (1). In another embodiment of the present invention, rRNA removal can be applied to ssDNA obtained in the step (4) or to dsDNA amplified by PCR in the step (5). When rRNA is removed after obtaining dsDNA, for example, CRISPRclean Plus Stranded Total RNA Prep with rRNA Depletion (Human, Mouse, Rat, Pan Bacteria) (JUMPCODE GENOMICS) can be used.

In one embodiment, the method for preparing a DNA library from a sample containing RNA of the present invention can be multiplexed from the lysate preparation stage. For example, lysates can be prepared from multiple different samples simultaneously and in parallel, for example, by using a multi-well plate (e.g., 96-well microplate, 384-well microplate etc.) to prepare libraries. According to the present invention, a next-generation sequencing cDNA library can be prepared from a small amount of sample, e.g., a lysate of less than 1×10⁵, less than 5×10⁴, less than 1×10⁴, less than 5×10³, or less than 1×10³ cells, and sequencing and/or quantification can be performed with a low bias. Since lysis of a small amount of cells can be performed in a volume of, for example, 400 μL or smaller, a multi-well microplate can be used to prepare lysates. For the multiplexed preparation of libraries from lysates, a different sample identifier can be added to each sample, and thereby the origin of the sample can be determined. As for the sequencing and/or quantification of libraries created from multiplexed lysates, high-throughput sequencing can be performed collectively (performed for pooled samples). In such cases, a different sample identifier can be added to each sample, and thereby the origin of the sample can be determined.

In one embodiment of the present invention, the method for preparing a DNA library from a sample containing RNA can be used to prepare multiple samples in parallel. For example, a multi-well plate can be used to create cDNA libraries for ribosome profiling from multiple samples in parallel. The conventional Ribo-Seq can simultaneously perform experiments by the ribosome profiling method for only about 8 samples due to technical limitations. The method of the present invention can generate a cDNA library for ribosome profiling from a small amount of sample (e.g., RNA material of about 10³ to 10⁶ cells) without compromising the quality of the data. Therefore, as shown in the section of Examples mentioned later, the ribosome profiling method can be performed even from a small amount of cells (e.g., about 10⁴ cells) on a multi-well plate (e.g., 96-well plate or 384-well plate). That is, the method of the present invention can be performed as a multisampling method in which all samples on a multi-well plate are tested simultaneously in parallel. The method of the present invention (Thor-Ribo-Seq) can be performed as a high-throughput method (HT-Thor-Ribo-Seq) by utilizing a multi-well plate and an automated pipetting system, e.g., ASSIST PLUS (INTEGRA Biosciences). The method of the present invention can also be performed on a part of intracellular organelles purified. The method of the present invention can be used to analyze intramitochondrial translation at one codon resolution. For example, by applying Thor-Ribo-Seq to concentrated mitochondrial ribosomes obtained from pre-purified mitochondria, intramitochondrial translation can be analyzed at one codon resolution.

In one embodiment, the method for preparing a DNA library from a sample containing RNA of the present invention can be performed to analyze mitochondrial translation. Mitochondrial translation refers to translation that takes place within mitochondria. In addition to the cytoplasmic translation system, eukaryotic cells have a dedicated translation system within the mitochondria. Although this system translates only 13 different mRNAs, but each of which encodes an essential factor for the oxidative phosphorylation complex, and therefore it is an extremely important translation system for organisms. Elucidation of mitochondrial translation dynamics remains as a major challenge, and to achieve this object, a technique for performing a low-input ribosome profiling method with scalability to expand to multiple samples has been needed. As shown in the section of Examples described below, the method of the present invention can create a library for ribosome profiling from a small amount of sample (e.g., RNA material of about 10³ to 10⁶ cells) without compromising the quality of the data. Furthermore, as mentioned above, the method of the present invention can be used to prepare multiple samples in parallel. Therefore, the method of the present invention will be useful for elucidating mitochondrial translation dynamics.

The method for preparing a DNA library from a sample containing RNA of the present invention can be applied to search for therapeutic agents for various diseases. For example, by combining chemical screening with high-throughput HT-Thor-Ribo-Seq, compounds that inhibit or activate the synthesis of mitochondrial disease-associated proteins, while covering the activities of intracytoplasmic and intramitochondrial translations.

### <Kit>

The present invention relates to a kit for performing a method for preparing a DNA library from a sample containing RNA or a method for sequencing and/or quantifying an RNA molecule in a sample containing RNA, which comprises:
a first DNA linker containing at least a promoter antisense sequence,
a first oligonucleotide containing at least a promoter sense sequence; and
a second DNA linker.

In a specific embodiment of the present invention, the kit further comprises: an RNA polymerase that recognizes the promoter sequence; and/or
a reverse transcriptase; and/or
a second oligonucleotide used as a primer for reverse transcription; and/or
a pair of primer oligonucleotides for PCR.

The first DNA linker of the present invention comprises at least a promoter antisense sequence.

In a specific embodiment of the present invention, the first DNA linker can contain a sequence necessary for next-generation sequencing analysis, such as a sequence providing sequencing primer binding site, and nucleotides constituting flow cell primer sequence and/or barcode (unique molecular identifier and sample identifier) sequence.

In a specific embodiment of the present invention, the first DNA linker can contain any of the following sequences:

(SEQ ID NO: 1)
AGATCGGAAGAGCACACGTCTGAACTCCAGTCACATTATCCCTATA
GTGAGTCGTATTAAATTC,
and
(SEQ ID NO: 2)
AGATCGGAAGAGCACACGTCTGAACTCCAGTCACCCTATAGTGAGT
CGTATTAGTCA.

The first DNA linker can further contain a barcode sequence, i.e., a sequence for a unique molecular identifier (UMI) and/or a sample identifier sequence.

The first oligonucleotide of the present invention can hybridize to the 3′ end portion of the first linker. In one embodiment of the present invention, the first oligonucleotide comprises at least a promoter sense sequence. In a specific embodiment of the present invention, the first oligonucleotide comprises the following sequences:

(SEQ ID NO: 3)
TGACTAATACGACTCACTATAGGGTGACTGGAGTTCAG,
and
(SEQ ID NO: 4)
GAATTTAATACGACTCACTATAGGGATAATGTGACTGGAGTTCAG.

In a specific embodiment of the present invention, the second DNA linker used in the method of the present invention comprises the following sequence:

| (SEQ ID NO: 5) | |
|---|---|
| | AGATCGGAAGAGCGTCGTGTAGGGAAAGAG |

In one embodiment of the present invention, the second DNA linker can further contain a random sequence as a barcode sequence, i.e., a sequence for a unique molecular identifier (UMI) and/or a sample identifier sequence.

In a specific embodiment of the present invention, the second oligonucleotide used as the primer for reverse transcription in the step (4) mentioned above comprises the following sequence:

(SEQ ID NO: 10)
AATGATACGGCGACCACCGAGATCTACACTCTTTCCCTACACGACGCTC

The 5′ end of the primer for reverse transcription can be phosphorylated (SEQ ID NO: 29).

In a specific embodiment of the present invention, the pair of primer oligonucleotides for PCR consists of the second oligonucleotide and the third oligonucleotide.

In a specific embodiment of the present invention, the second oligonucleotide comprises the sequence shown below:

(SEQ ID NO: 10)
AATGATACGGCGACCACCGAGATCTACACTCTTTCCCTACACGACGC
TC

In a specific embodiment of the present invention, the third oligonucleotide comprises the nucleotide sequence shown below:

(SEQ ID NO: 11)
CAAGCAGAAGACGGCATACGAGATCGTGATGTGACTGGAGTTCAGACGTG
TG

Flow cell primers can be incorporated into the cDNAs of the DNA library by PCR using the second and third oligonucleotides.

In addition to the above, the kit of the present invention can include a container (e.g., tube, plate, multi-well plate (e.g., 96-well plate), dish etc.), lysis buffer (e.g., for cell lysis), buffer for RNA extraction, other buffers (various buffers suitable for enzymes for ligation, in vitro transcription, reverse transcription, PCR and others), water, salts (MgCl₂, etc.), enzyme (proteinase K, nuclease, etc.), magnetic beads (e.g., for rRNA removal, RNA purification, etc.), glycerol, surfactant (10% SDS, etc.), polymer (mPEG, PEG8000, etc.), dNTPs, GTP, ATP, DTT, control, size markers (for RNA and for DNA), probe, instructions for use, labels, etc.

Although 96 examples of the sample identifier are listed in the following table, sample identifiers that can be used in the present invention are not limited to these.

TABLE 1

| Sequence (5′->3′) | SEQ ID NO: |
|---|---|
| CATATTCCTGGTGG | 30 |
| ACAACAAGGTTCGG | 31 |
| TCGGTGAGGATTGG | 32 |
| GACAACATCAGCGG | 33 |
| ATATTGCAACCTGG | 34 |
| TTGGAACCTTGCCG | 35 |
| CCTTCTCCATCCTG | 36 |
| CGCGAATTGATTAG | 37 |
| TTAGGCGTTGGTGG | 38 |
| ACAAGTCTCACGTG | 39 |
| AGACGGAGTTGTGG | 40 |
| ACATGAGTGTTCTG | 41 |
| ATATGAAGGCTTGG | 42 |
| CCTTAGATCCTCCG | 43 |
| TACATAAGCCTTCG | 44 |
| GCCAGGAAGTCTTG | 45 |
| AGCAAGACTAGCGG | 46 |
| AGCCTTGGATCTGG | 47 |
| GAGGCTTAACTTGG | 48 |
| TGACAGGACTTCGG | 49 |
| GGAGCACTAGCTTG | 50 |
| ATGTTATCTCGTGG | 51 |
| AGCTAATGTTGTCG | 52 |
| GGAACGATATTCAG | 53 |
| TGCTGTCAGATTGC | 54 |
| CTATTGCCTTCGCC | 55 |
| CAGAGGCTGTTGTC | 56 |
| ATGAGTAGACCTTC | 57 |
| ACCGTTAAGTCGCC | 58 |
| ATCTGTGGTTCTAC | 59 |
| TGTGCGGTTCGTTC | 60 |
| AAGCGCCATGGTTC | 61 |
| TCAGAGGTAGTTAC | 62 |
| GTTGGTTAGTCTTC | 63 |
| CATTACTATACCGC | 64 |
| CGACAACTACTGCC | 65 |
| AACCACGATAGGCC | 66 |
| GCTAGAGACTCTTC | 67 |
| GTTAGGAAGTTCAC | 68 |
| TTGATCCGTTCTTC | 69 |
| GGTTAGTAGATTCC | 70 |
| CCGGACTAACCTTC | 71 |
| AGCATGGACTCTCC | 72 |
| TTCCAGATACCTTC | 73 |
| ATAAGACACTCTGC | 74 |
| GAATTCTCGATTCC | 75 |
| GTCAATTCACCTAC | 76 |
| GGTATTAAGACGTC | 77 |
| AATATCTTCGGCCA | 78 |
| AGTGGCGTTAACAA | 79 |
| GTGTCCGGCATTGA | 80 |
| CAACCGGAGAAGAA | 81 |
| CTCAGGACTTAGTA | 82 |
| GCTTAGACTAGCTA | 83 |
| ACATTGTCCACCAA | 84 |
| ATCGCGCCAACCAA | 85 |
| GCGAAGTCCGAGAA | 86 |
| ATTAGGAGCTCGAA | 87 |
| GCGTGACGCAGTAA | 88 |
| GGATCCTTAGACAA | 89 |
| GAAGCATAGGAGAA | 90 |
| CACCTCCATGTGTA | 91 |
| CGCATGCTATAGAA | 92 |
| TGGTCGATCTTGAA | 93 |
| TAGATCTCGCAGAA | 94 |
| TAGTGAGCTAGTCA | 95 |
| ATGAGCGCATATCA | 96 |
| CTATACAGTGCGTA | 97 |
| GAGAGAAGGTTGTA | 98 |
| CTGGAGAGGTACAA | 99 |
| TTATACGATCGCCA | 100 |
| CTCGAATCCTACAA | 101 |
| AACAGACCGTTACA | 102 |
| GTGCCTCACCGATA | 103 |
| TTGCGCCTCACATA | 104 |
| ACCGCGAAGATATA | 105 |
| CAGGACCGTATATA | 106 |
| TTATCTAGACGAGG | 107 |
| ACTATCGCGCTAGG | 108 |
| ATTGGAGCATTAGG | 109 |
| ATGCAGAGCTGACG | 110 |
| ATTGTATCACCACG | 111 |
| GTAAGTCCTATACG | 112 |
| TTCATGACCAGATG | 113 |
| GACAGGAGAGAGAT | 114 |
| CCAGTCCAACAGAT | 115 |
| CAGAATGGACAGAT | 116 |
| GTTCGCAGGCAGAT | 117 |
| CATGCGAGTCGGAT | 118 |
| GGAACGAGGACGAT | 119 |
| CGGAAGCCACTTAT | 120 |
| AGTCCACTAGGCGT | 121 |
| TTGGTCTGGTGCGT | 122 |
| CTCACCGTGATCGT | 123 |
| TAACAAGTGCTCGT | 124 |
| GGATTCCAGTTCGT | 125 |

In one embodiment of the present invention, UMI can be incorporated into the first DNA linker and the second DNA linker. When UMI is incorporated into the first DNA linker and the second DNA linker, the total length of the UMI can be 5 to 10 nt.

### EXAMPLES

The present invention will be explained more specifically with reference to the following examples, but the present invention is not limited to these examples.

### Example 1

### <Experiment 1>: Preparation of Cell Lysate

### Experimental Methods

### Cell Line

Flp-In T-REX 293 cells (Thermo Scientific, R78007) were cultured in DMEM, high glucose, GlutaMAX Supplement (Thermo Scientific) with 10% FBS at 37° C. and 5% CO₂ according to the manufacturer's instructions. The cell culture was routinely tested for Mycoplasma contamination using e-Myco VALID Mycoplasma PCR Detection Kit (iNtRON Biotechnology) to confirm the negativity.

### Construction of Library for Ribo-Seq

### Preparation of Cell Lysate

Cell lysate was prepared as descried in Reference 8 (a list of references is provided at the end of this description, the same applies below). Briefly, Flp-In T-REx 293 cells cultured in a 10-cm dish were washed with ice-cold PBS, and lysed with a lysis buffer [20 mM Tris-HCl pH 7.5, 150 mM NaCl, 5 mM MgCl₂, 1 mM dithiothreitol (DTT), 1% Triton X-100, 100 μg/ml cycloheximide and 100 μg/ml chloramphenicol]. The extract was treated with 0.025 U/μl Turbo DNase (Thermo Fisher Scientific), clarified by centrifugation at 20,000×g and 4° C. for 10 minutes, flash frozen in liquid nitrogen, and stored at −80° C. The RNA concentration in the lysate was measured with Qubit RNA BR Assay Kit (Thermo Fisher Scientific). Note that in standard Ribo-Seq, cells were treated with DMSO for 15 minutes.

### <Comparative Example> Standard Ribo-Seq

Standard Ribo-Seq was performed as explained in Reference 8. The cells containing RNA in an amount of 10 μg were suspended in the lysis buffer a volume of 300 μL and treated with 20 U (i.e. 0.067 U/μl) of RNase I (LGC Biosearch Technologies) at 25° C. for 45 minutes. The cells were then treated with 200 U of SUPERase-In RNase Inhibitor (Thermo Fisher Scientific) on ice and ultracentrifuged in a sucrose cushion at 100,000 rpm and 4° C. for 1 hour with a TLA110 rotor (Beckman Coulter). The ribosome pellet was dissolved in TRIzol reagent (Thermo Fisher Scientific) and RNA was isolated by using Direct-zol Research. The portion corresponding to RNA fragments of 17 to 34 nt in length was excised from the gel, the fragments were dephosphorylated with T4 polynucleotide kinase (New England Biolabs), and ligated to the linker with T4RNA Ligase 2, truncated KQ (New England Biolabs), rRNA was removed by using Ribo-Zero Gold rRNA Removal Kit (Human/Mouse/Rat, Illumina), and reverse transcription was performed by using ProtoScript II Reverse Transcriptase (New England Biolabs). The cDNA was then cyclized with CircLigase II ssDNA ligase (LGC Biosearch Technologies) and amplified by PCR using Phusion High-Fidelity DNA Polymerase (New England Biolabs). The DNA library was sequenced by using HiSeq 4000 (Illumina) with a single-ended 50 bp option.

### <Example> Method of the Present Invention (Thor-Ribo-Seq)

For the “aliquoted-then-digested” experiments, 1, 0.1, and 0.01 μg of RNA was scaled up to 300 μl with the lysis buffer, digested with 0.067 U/μl RNase I at 25° C. for 45 minutes, placed on ice, supplemented with 200 U SUPERase-In RNase Inhibitor, and subjected to sucrose cushion.

For the “digested-then-aliquoted” experiments, the cell lysate was treated with RNase I and SUPERase-In Inhibitor as described for the standard Ribo-Seq (method of Comparative Example). Then, aliquots corresponding to 1, 0.1, and 0.01 μg of RNA were made up to a volume of 300 μl with the lysis buffer and applied to a sucrose cushion.

### <Experiment 2> Preparation of cDNA from RNA

RNA isolation and gel excision were performed as previously described (Reference 8).

The following linkers and oligonucleotides were used.

### First DNA Linker (SI181)

(SEQ ID NO: 12)
5′-/Phos/NNNNNATCGTAGATCGGAAGAGCACACGTCTGAACTCCAGT
CACCCTATAGTGAGTCGTATTAGTCA/ddC/-3′,

where/Phos/represents 5′ phosphate,/ddC/represents terminal 2′,3′-dideoxycytidine, N represents a random nucleotide for UMI, bold characters represent a sample identifier, and the underlined portion represents the T7 promoter antisense sequence,

As an alternative for the first DNA linker, the following can also be used SI96_001

(SEQ ID NO: 13)
5′-/Phos/NNNNNCATATTCCTGGTGGAGATCGGAAGAGCACACGTCTG
AACTCCAGTCACATTATCCCTATAGTGAGTCGTATTAAATTC/SpC3/-
3′,

where/Phos/represents 5′ phosphate,/SpC3/represents 3′ C3 spacer, N represents a random nucleotide for UMI, bold characters represent a sample identifier, and the underlined portion represents the T7 promoter antisense sequence and a transcription activity-increasing sequence added to the flanking region of the antisense sequence.

### Second DNA Linker (SI183)

(SEQ ID NO: 14)
5′-/Phos/NNAGATCGGAAGAGCGTCGTGTAGGGAAAGAG/ddC/-3′

First oligonucleotide (T7 promoter oligonucleotide) (SI182)

(SEQ ID NO: 3)

5′-TGACTAATACGACTCACTATAGGGTGACTGGAGTTCAG-3′,

where the underlined portion represents the T7 promoter sense sequence.

As an alternative for the first oligonucleotide, the following can also be used along with the alternative first DNA linker (SI96_001)

SI194
(SEQ ID NO: 4)
5′-GAATTTAATACGACTCACTATAGGGATAATGTGACTGGAGTTCAG-
3′,

where the underlined portion represents the T7 promoter sense sequence and a transcription activity-increasing sequence added to the flanking region of the sense sequence.

### Second Oligonucleotide (SI186) (Primer for Reverse Transcription)

(SEQ ID NO: 29)
5′-/Phos/AATGATACGGCGACCACCGAGATCTACACTCTTTCCCTACA
CGACGCTC-3′

### Second and Third Oligonucleotides (PCR Primers NI798 and NI799)

(SEQ ID NO: 10)
5′-AATGATACGGCGACCACCGAGATCTACACTCTTTCCCTACACGACGC
TC-3′
(SEQ ID NO: 11)
5′-CAAGCAGAAGACGGCATACGAGATCGTGATGTGACTGGAGTTCAGAC
GTGTG-3′

The first DNA linker (SI181) and the second DNA linker (SI183) were pre-adenylated with Mth RNA Ligase (New England Biolabs) as previously described (Reference 8).

After dephosphorylation with T4 polynucleotide kinase, RNA was ligated with the preadenylated first DNA linker SI181 by using T4 RNA Ligase 2, truncated KQ.

After hybridization of the T7 promoter oligonucleotide SI182, complementary RNA was transcribed by using T7-Scribe Standard RNA IVT Kit (CELLSCRIPT) at 37° C. for 2.5 hours and gel purification was performed. After dephosphorylation with T4 polynucleotide kinase, RNA was ligated to the preadenylated second linker SI183 by using T4 RNA Ligase 2, truncated KQ, hybridized to the second oligonucleotide (SI186) and then reverse-transcribed by using ProtoScript II Reverse Transcriptase. cDNA was PCR-amplified with the PCR primers NI798 and NI799 by using Phusion High-Fidelity DNA Polymerase.

rRNA removal using the Rib-Zero unit of TruSeq Stranded Total RNA Library Prep Gold was applied to cDNA, but it can also be applied to RNA that has been linked to the first DNA linker.

The DNA library was sequenced with HiSeq X (Illumina) at a paired-end of 150 bp.

### <Experiment 3> Deep Sequencing Data Analysis

Data processing was performed as previously described in Reference 7 with modifications. By using Fastp v0.21.0 (Reference 49), bases of pair-end reads were corrected by overlap analysis, and then quality filtering and removal of the adapter sequence (5′-AGATCGGAGAGCACACGTCTGA-3′, SEQ ID NO: 15) were performed on read 1. The algorithm for trimming adapter sequences removes all downstream sequences once this sequence is detected. For single-end reads, base correction was skipped. After the extraction of UMI and barcode sequences on the linkers by a custom script (fastx-split in https://github.com/ingolia-lab/RiboSeq), reads mapped to noncoding RNA were excluded from the analysis by using STAR v2.7.0a (Reference 50). By using STAR, the remaining reads were aligned to the human genome hg38 and assigned to the GENCODE Human release 32 reference. UMI-based deduplication was performed by using UMI tools v1.1.2 (Reference 51). Downsampling of the data set was performed by using seqtk (https:/github.com/lh3/seqtk). The definition of representative genes was based on MANE selectv.1.0 (Reference 52).

In the standard Ribo-Seq (the method of Comparative Example), the A-site offsets were determined to be 15 for footprints of 20 to 22 and 24 to 31 nt and 16 for footprints of 23 and 32 nt, based on the metagene analysis of the 5′-ends of footprints. In Thor-Ribo-Seq (the method of the present invention), the A-site offsets were defined to be 15 for footprints of 21, 22 and 27 to 31 nt and 16 for footprints of 23, 32 and 33 nt. Ribosome occupancies were defined as the number of reads at a given codon, normalized by the mean number of reads per codon on each CDS. Transcripts with 1 or more reads per codon on average in all samples were included in the analysis.

### Resource Availability

Standard Ribo-Seq and Thor-Ribo-Seq data (GEO: GSE222195) were deposited in the National Center for Biotechnology Information (NCBI) database.

### (Results)

Implementation of RNA-Dependent RNA Amplification into Ribo-Seq

An apparent pitfall of Ribo-Seq is the necessary material amount or cell number for library preparation. In fact, standard ribosome profiling experiments (FIG. 1-1, A) typically require 1 to 10 μg of total RNA, which corresponds to approximately 10⁵ to 10⁶ cells of human embryonic kidney (HEK) 293. Smaller quantities of cell lysate, such as extract corresponding to 0.1 μg of total RNA (or about 10⁴ cells), could not enable DNA library amplification by PCR (FIG. 1-1, C) due to sample loss during the multiple complicated steps in the library preparation. This drawback ultimately restricted ribosome profiling experiments for limited samples.

To overcome this problem, the inventors of the present invention successfully amplified linker ribosome footprints at the early stage of the library preparation (FIG. 1-1, B; FIG. 2-2, A) before material loss. In the present invention, the linker DNA oligonucleotides were designed on the basis of the antisense sequence of the T7 promoter. Hybridization of short DNA oligonucleotide covering the antisense sequence formed a partial dsDNA region suitable for RNA transcription (Reference 24). T7 polymerase can synthesize complementary RNA from an RNA template conjugated to the dsDNA T7 promoter region (Reference 25), and therefore this configuration can be used to amplify complementary RNA of ribosome footprint. Further, optimized DNA sequences were employed upstream and downstream of the T7 promoter to maximize the transcription (Reference 26). For multiplexing, the linker also has a sample barcode sequence (FIG. 1-1, B). To suppress amplification bias, a unique molecular index (UMI) was added to the linkers (FIG. 1-1, B). The Thor (T7 High-resolution Original RNA) strategy, a highly sensitive RNA analysis method using T7 RNA polymerase, is implemented in LUTHOR 3′ mRNA-Seq Library Prep Kit provided by Lexogen (Reference 53), but LUTHOR adds a double-stranded T7 promoter by reverse transcription and end repair, which is different from the present invention.

In this example, the method of the present invention (Thor-Ribo-Seq) was compared with the standard Ribo-Seq for the performance using normal amounts of material (10 μg of total RNA or about 10⁶ cells). In Thor-Ribo-Seq of the present invention, as in the standard Ribo-Seq, hallmarks of the ribosome footprints, peaks of reads at 22 nt and 28 nt (FIG. 3, A) and strong triplet periodicity along CDS (CoDing Sequence) (FIG. 3, B and C) were observed. Both Ribo-Seq methods also detected a similar number of genes (FIG. 3, D). Thus, high correspondence of reads on ORFs was found between the standard Ribo-Seq and Thor-Ribo-Seq (FIG. 2-2, B).

Furthermore, the method of the present invention (Thor-Ribo-Seq) suffered from limited artifacts in the codon-wise examination of ribosome occupancy. The codon sequences on the ribosomal A site were the most prominent determinant of ribosome duration time at each codon (References 27 to 36), and thus differences in ribosome occupancy are shown (FIG. 2-2, C). The ribosome occupancy divergence was consistently maintained in the method of the present invention (Thor-Ribo-Seq) (FIG. 2-2, C). These results indicate that the Thor-Ribo-Seq strategy provides an option with as few experimental artifacts as possible, at least for conventional sample amounts.

Translation Profiling from Low Material Input by Thor-Ribo-Seq

The Thor-Ribo-Seq strategy of the present invention surprisingly makes it possible to perform ribosome profiling with inputs too low to be processed by the standard Ribo-Seq (small amounts of RNA material). Considering that RNase treatment (References 10, and 37 to 39) and downstream steps (References 29 and 33) introduce different biases, experiments to separately control the two impactful sources of influence were planned when applying Thor-Ribo-Seq to small amounts of RNA material. To maintain the same RNase treatment conditions, a library was created with an aliquot of RNase-treated lysate (described as “digested-then-aliquoted”) (FIG. 4-1, A). A cell lysate containing 10 μg of total RNA (or about 10⁶ cells) was treated with RNase I as in the standard ribosome profiling, and then the reaction mixture was divided into aliquots corresponding to 0.01, 0.1, and 1 μg of total RNA for the downstream Thor-Ribo-Seq library preparation. Transcription with T7 polymerase allowed amplification of complementary RNA (FIG. 4, B), and amplification of the sequencing library (FIG. 4-1, C) with at least 0.01 μg (corresponding to about 10³ cells) of input (material). In the sequencing data, benchmarks of footprints, including read length (FIGS. 5-1, A) and 3-nt periodicity (FIG. 5-2, B to D), were maintained.

Further, in this example, the translation status of the entire transcriptomes was assessed in the “digested-then-aliquoted” Thor-Ribo-Seq experiment. The reads on the ORFs showed high correlations with Thor-Ribo-Seq data generated from about 10⁶ cells (FIG. 5-3, I). Furthermore, the improved method of the present invention yielded ribosome footprints from a large number of genes (FIG. 5-3, J), suggesting only a slight limitation in detection sensitivity. The slight decrease at minimal input (about 10⁴ cells) may be due to the shallow complexity of the transcripts in the lysate. This is considered to be because, for example, mRNA of which amount is extremely small within cells (e.g., expressed in only a part of cells) is less likely to be present in the sample when the original number of cells in the lysate becomes smaller. In addition to the mRNA-wise analysis, the approach of the method of the present invention also allowed codon-wise analysis of ribosomal occupancy from low inputs (FIG. 5-3, K). These data showed that even if there is any bias due to the procedural steps after RNase digestion, it does not interfere with the ThorRibo-Seq translatome analysis for small amounts of input.

The inventors of the present invention noted that the suppression of read duplications generated by T7 polymerase-mediated RNA amplification benefited the adequate evaluation of data. The Thor-Ribo-Seq library prepared by the method of the present invention contained unique molecular indexes (UMIs) at two distinct positions: the 3′ end of footprint introduced by the first linker and the 5′ end of footprint originating from the second linker (FIG. 2-2, B and FIG. 5-4, L). Given the timing of the addition of UMI, the 3′ end UMI suppressed the read duplications that occurred in the in vitro transcription, and the combination of 3′ and 5′ UMIs limited duplications of them in the PCR (FIG. 5-4, L). The potency of UMI to restrain the biases was tested, and found that the minimization of overamplified reads by in vitro transcription led to a high correlation among Thor-Ribo-Seq data compared with the same correction of biases in PCR (FIG. 5-3, I and FIG. 5-4, M). In the above analyses, 3′ UMI-based duplication suppression was used unless otherwise noted.

The inventors of the present invention developed Thor-Ribo-Seq as a sensitive method for translatome analysis with limited materials while maintaining the characteristics of ribosome profiling experiments. The strategy of the method of the present invention outperforms in A-site offset assignments than the existing methods for low inputs (small amounts of RNA material). Micrococcal nuclease (MNase) used in ScRibo-Seq (Reference 13) is known to have a preference for A/U due to stringent activity control by Ca²⁺ ions. The poly (A) tailing and template switching used in one-pot reactions (References 9 to 12) add homopolymer nucleotides to the footprints, thus generating ill-defined read boundaries at both the 5′ and 3′ ends. These drawbacks hamper the A-site offset estimation of ribosome footprints. In contrast, the strategy of the inventors of the present invention uses less biased RNase I and double-linker ligation to explicitly determine the ends of the reads, preventing ambiguity regarding the A-site positional reference.

Although the method of the present invention requires custom-made linkers, all other reagents are commercially available. Therefore, the method is not only used for ordinary ribosome profiling, but also easily introduced for library construction based on selective ribosome profiling (References 40 and 41), translation complex profile sequencing (TCP-Seq or 40S footprinting, References 42 and 43), selective TCP-Seq (References 44 to 47), cross-linking and immunoprecipitation (CLIP)-Seq (Reference 48), etc. All of them should enable deep sequencing using limited amounts of RNA fragments.

### Example 2

In this example, to investigate the translational landscape under microgravity, genome-wide ribosome profiling was performed on HEK293 cells cultured in the International Space Station (ISS) (FIG. 7, A). For this purpose, the cells were first cultured under 1×g artificially generated by centrifugation in the ISS, and then cultured under microgravity for 24 and 48 hours (FIG. 7, A). Control cells, on the other hand, were maintained at 1×g until collection. The cells were treated with translation inhibitors (cycloheximide and chloramphenicol), frozen, and returned to the ground laboratory for library preparation. Constraints of space flight limited the materials available for standard ribosome profiling experiments. To overcome this problem, Thor-Ribo-Seq was used in this example.

The specific experimental method is described in Reference 54. In the space flight experiment, culture was performed in a disposable culture chamber (DCC, JAX A, Chiyoda Corporation) with a 3D clinostat (5.5 rpm for X axis, 6.5 rpm for Y axis, AES) and centrifuge (AES). To prevent shear stress due to air bubbles, DCC was completely filled with the medium. DCC was pretreated overnight with laminin. Then, 5×10⁵ HEK293 cells were seeded into each DCC and incubated overnight. The medium was replaced with CELLBANK ER 1 plus (TaKaRa) and the culture was stored at −80° C. The frozen cells were launched to the ISS by Cygnus NG14 in October 2020. In the Japanese Experiment Module, Kibo, of the ISS, they were thawed, the medium was replaced with 10% FBS-supplemented DMEM, high glucose, GlutaMAX Supplement (Thermo Fisher Scientific) by using Pre-Fixation Kit-III (PFKIII, JAXA), and culture was performed under centrifugation at 1×g, 37° C., 5% CO₂, and 80±5% RH for 24 hours in the Cell Biology Experiment Facility (CBEF). The cells were then cultured under microgravity for 24 and 48 hours, while the control cells were maintained at 1×g. For chemical fixation, the cell culture medium was replaced with CELLBANKE R 1 plus containing 100 μg/ml cycloheximide and 100 μg/ml chloramphenicol, and fixation was performed with PFK-III. Then, the cells were stored in Minus Eighty degree Celsius Laboratory Freezer (MELFI) for ISS at −95° C. The samples were returned to the ground in July 2021 by SpaceX CRS22 (SpX22) in frozen condition and stored at −80° C. in the laboratory for ribosome profiling and RNA-Seq.

### Lysate Preparation

### HEK293 Cells

The cells in the DCC were thawed, washed with ice-cold PBS, and triturated 10 times in 500 μl of lysis buffer (20 mM Tris-HCl pH 7.5, 150 mM NaCl, 5 mM MgCl₂, 1 mM dithiothreitol [DTT], 1% Triton X-100, 100 μg/ml cycloheximide, and 100 μg/ml chloramphenicol) with a syringe having a 30 gauge needle (Nipro Corporation). The lysate was incubated with 25 U/ml TURBO DNase (Thermo Fisher Scientific) and clarified by centrifugation at 20,000×g and 4° C. for 10 minutes.

### Nematode

Frozen nematode cultures (1.5 to 4.5 ml) were each thawed in 10 ml of wash buffer (20 mM Tris-HCl pH 7.5, 150 mM NaCl, 5 mM MgCl₂, 100 μg/ml cycloheximide, and 100 μg/ml chloramphenicol). Adult worms were taken by filtration through 30-μm UberStrainer (pluriSelect Life Science) and washed three times with the wash buffer. The nematodes were then pelleted by centrifugation at 1,600×g and 4° C. for 1 minute, resuspended in 600 μl of the lysis buffer, and dripped into liquid nitrogen. They were crushed in Multi-beads Shocker at 2800 rpm for 10 seconds, thawed at 4° C., and incubated with 25 U/ml Turbo DNase (Thermo Fisher Scientific), and the suspension was clarified by centrifugation at 20,000×g and 4° C. for 10 minutes.

### Preparation of Library for Ribosome Profiling

The lysate was treated with 20 U of RNase I (LGC Biosearch Technologies) at 25° C. for 45 minutes. Ribosomes were collected by using sucrose cushion. The RNA was electrophoresed on 15% UREA PAGE gel. rRNA was removed by using Ribo-Zero Gold rRNA Removal Kit (Human/Mouse/Rat, Illumina) for the human and mouse samples, and by using Caenorhabditis elegans Ribo-Seq riboPOOLs (siTOOLS Biotech) for the nematode samples. The sample was hybridized with the oligonucleotide at the T7 promoter region of the linker, and then a complementary RNA was transcribed with T7-Scribe Standard RNA IVT Kit (CELLSCRIPT) and ligated to the second DNA linker. The cDNA was reverse transcribed and PCR-amplified. The DNA library was sequenced on HiSeq 4000 in the 50 bp single read mode (s-μg experiment) or HiSeq X Ten in the 150 bp paired-end mode.

### Preparation of Library for RNA-Seq

For RNA-seq, total RNA was extracted from the lysate used for the ribosome profiling by using TRIzol LS (Thermo Fisher Scientific) and Direct-zol RNA Microprep Kit (Zymo Research). rRNA was removed by using Ribo-Zero Gold rRNA Removal Kit (Human/Mouse/Rat, Illumina) for the human and mouse samples, and by using Caenorhabditis elegans Ribo-Seq riboPOOLs (siTOOLs Biotech) for the nematode samples. For the HEK293 cells for space flight experiments, a library was prepared with SMAR Ter Stranded Total RNA-Seq Kit v3-Pico Input (TaKaRa). The DNA library was sequenced on HiSeq 4000 in the 50 bp single sequenced mode (s-μg experiment) or on HiSeq X Ten in the 150 bp paired-end mode (other experiments). The data analysis was as described in Reference 54.

### Results

In this example, Thor-Ribo-Seq allowed to obtain high quality data on samples returned from the ISS, confirming a sharp peak of read length at 28 or 29 nt, (FIG. 8-1, A), which is a characteristic of the ribosome footprints, 3 nt periodicity (FIG. 8-1, B), and high reproducibility (FIG. 8-1, C left). The results revealed the effects of microgravity on translation, especially the reduction of some mRNAs. Among them, protein synthesis from mitochondrial mRNAs encoded by the organelle genome was found to be significantly sensitive to microgravity (FIG. 7, B, left; FIG. 8-2, D, left; FIG. 8-3, E). The method of the present invention allows obtaining footprints from both cytoplasmic ribosomes (cytoribosomes) and mitochondrial ribosomes (mitoribosomes), making it possible to investigate the both translation systems simultaneously. It was found that the decrease in the footprints of mitoribosomes could not be explained by the decrease in the corresponding mRNA measured by RNA sequencing (RNA-Seq) of the same material (FIG. 7, B, middle; FIG. 8-1, C, right; FIG. 8-2, D, middle; FIG. 8-3, E). Thus, the translation efficiency calculated on the basis of the excess or deficiency of the ribosome footprints relative to the RNA-Seq fragment still showed a decrease in mitochondrial mRNAs (FIG. 7, B, right). In contrast, translation of mitochondria-localized proteins encoded in the nuclear genome was insensitive to microgravity (FIG. 8-4, F and FIG. 8-4, G). Mitochondrial translation regulation may be an early response to microgravity. Over the time course of microgravity culture, changes in mitochondrial translation were observed to be more dramatic in 24 hours of microgravity culture than 48 hours (FIG. 7, C).

To further extend the search from cell culture to the whole body level, nematode samples flown on the ISS were used. The L1 larvae were cultured under microgravity for 4 days, while control worms were incubated in a 1×g centrifuge (FIG. 7, D). As in human cells, ribosome profiling and RNA-Seq of nematodes revealed translational changes in a subset of mRNAs. Surprisingly, a reduction in mitochondrial translation efficiency was observed, as found in the cell-based experiments (FIG. 7, E). Through these space flight experiments, it was concluded that the attenuation of mitochondrial translation is a response to microgravity, which is widely seen in higher eukaryotes. In addition, it was verified that the DNA library preparation method of the present invention successfully enables ribosome profiling analysis from rare space flight materials.

### Example 3

In this example, for the analysis of mitochondrial translation, the Thor-Ribo-Seq of the present invention and the conventional method of Ribo-Seq (MitoRibo-Seq) were compared. In the following, combination of the Thor-Ribo-Seq of the present invention and MitoIP, a mitochondrial immunoprecipitation method using anti-TOM22 antibody, is referred to as MitoIP-Thor-Ribo-Seq. The combination of the conventional method Ribo-Seq and MitoIP is called MitoIP-Ribo-Seq.

### Materials

As the cell lines. NSUN3 KO, GTPBP3 KO, QTRT1 KO, QTRT2 KO, OSGEPL1 KO HEK293 (References 56 to 60), wild-type or A3243G mutant myoblasts (derived from muscle specimens of MELAS patients), and naive HEK293 cells [American Type Culture Collection (ATCC), CRL-1573] were used.

### Lysate Preparation

Cells were cultured in a 15-cm dish, washed with ice-cold PBS, and then lysed with 1200 μL of a hypotonic buffer (10 mM HEPES-KOH PH 7.5, 10 mM KCl, 1.5 mM MgCl₂, 1 mM DTT, 100 μg/mL cycloheximide, and 100 μg/mL chloramphenicol). The lysate was incubated with beads conjugated with anti-TOM22 antibody of Mitochondria Isolation Kit, Human (Miltenyi Biotec) at 4° C. for 60 minutes. Subsequent washing and elution steps were performed by using wash buffer (10 mM HEPES-KOH PH 7.5, 10 mM KCl, 15 mM MgCl₂, 1 mM DTT, 100 μg/mL cycloheximide, and 100 μg/mL chloramphenicol) according to the manufacturer's instructions. The eluted mitochondrial fraction was pelleted by centrifugation at 7,000×g and 4° C. for 10 minutes and then resuspended in a modified lysis buffer (20 mM Tris-HCl pH 7.5, 150 mM NaCl, 15 mM MgCl₂, 1 mM DTT, 1% Triton X-100, 100 μg/mL cycloheximide, 100 μg/mL chloramphenicol, and 2.5 U/mL TURBO DNase). After clarification by centrifugation at 20,000×g and 4° C. for 10 minutes, the supernatant was used for library preparation.

### Library Preparation by MitoIP-Ribo-Seq

The cell lysate was incubated with 2 U of RNase I (LGC Biosearch Technologies, low), 10 U of RNase I (high), 0.12 U of RNase A/T1 (Thermo Fisher Scientific, low), or 0.6 U of RNase A/T1 (high) at 25° C. for 45 minutes in 50 μL of reaction mixture (scaled up with the modified lysis buffer). Then, for Ribo-FilterOut (Reference 61), mitoribosomes were pelleted with sucrose cushion and resuspended in an EDTA lysis buffer [20 mM Tris-HCl pH 7.5, 150 mM NaCl, 15 mM MgCl2, 1 mM DTT, 5 mM EDTA, 1% Triton X-100, and 20 U/ml SUPERase-In RNase inhibitor (Thermo Fisher Scientific)]. In this EDTA lysis buffer, EDTA is a chelating agent, which can detach mRNA from ribosomal subunits (WO2021/106814A1). The solution was loaded onto an Amicon Ultra 0.5 ml Ultracel 100K centrifugation filter (Millipore) and centrifuged at 14,000×g and 4° C. for 10 minutes (Reference 61). RNA from the flow-through was purified with TRIzol LS (Thermo Fisher Scientific) and Direct-zol RNA MicroPrep Kit (Zymo Research) and separated on a 15% UREA PAGE gel. Portions corresponding to fragments ranging from 17 to 50 nucleotides (nt) were excised from the gel. Subsequent library preparation followed a previous report (Mito et al., Non-patent document 8): the RNA fragments were dephosphorylated, and ligated with a linker, rRNA removal was performed by using Ribo-Zero Gold rRNA Removal Kit (Human/Mouse/Rat, Illumina, accompanied by TruSeq Stranded Total RNA Kit), cDNA was reverse-transcribed, circularized by ligation, and PCR-amplified. Sequencing of the DNA library was performed by using HiSeq 4000 in the 50 bp single read mode.

### Library Preparation by MitoIP-Thor-Ribo-Seq

The cell lysate was incubated with 40 U of RNase I (LGC Biosearch Technologies) in 50 μL of a reaction solution at 25° C. for 45 minutes. Then, for Ribo-FilterOut (Reference 61), mitoribosomes were pelleted with sucrose cushion and resuspended in the EDTA lysis buffer [20 mM Tris-HCl pH 7.5, 150 mM NaCl, 15 mM MgCl₂, 1 m M DTT, 5 mM EDTA, 1% Triton X-100, and 20 U/mL SUPERase-In RNase inhibitor (Thermo Fisher Scientific)]. The suspension was loaded onto an Amicon Ultra 0.5 mL Ultracel 100K centrifugation filter (Millipore) and centrifuged at 14,000×g and 4° C. for 10 minutes (Reference 61). RNA from the flow-through was purified with TRIzol LS (Thermo Fisher Scientific) and Direct-zol RNA MicroPrep Kit (Zymo Research) and separated on a 15% UREA PAGE gel.

Fragments ranging from 17 to 50 nt were recovered from monosomes.

A library was then prepared by Thor-Ribo-Seq according to “<Experiment 2> Preparation of cDNA from RNA” in Example 1. rRNAs were removed from the linker-bound RNA fragments using Human Ribo-Seq riboPOOL (siTOOLs Biotech). The oligonucleotide was then hybridized to the T7 promoter region of the linker to create dsDNA T7 promoter. A complementary RNA was transcribed with the T7-Scribe Standard RNA IVT kit (CELLSCRIPT). After ligation of the second linker, cDNA was reverse transcribed and PCR-amplified. The DNA library was sequenced on HiSeq X Ten in the 150-bp paired-end mode.

### Data Analysis

Deep sequencing data were processed according to the descriptions of Reference 62 and McGlincy, N. J. & Ingolia, N. T. 2017 (Non-patent document 7) with modifications. In pair-end sequencing, base correction was performed with fastp (version 0.21.0, Reference 49). Read quality filtering and adapter sequence removal were performed with fastp. All the reads were aligned with non-coding RNAs (rRNA, tRNA, mt-rRNA, mt-tRNA, snRNA, snoRNA, and miRNA), and the remaining reads were aligned with human or mouse nuclear genome (human, hg38; mouse, mm10) and mapped to a custom database of mitochondrial transcript sequences using STAR (version 2.7.0a, Reference 63). For MitoIP-Thor-Ribo-Seq, UMItools (version 1.1.2, Reference 64) was used for suppression of read duplication according to the unique molecular identifiers (UMI) of the linker.

The ribosomal A site offset for each footprint length was empirically estimated. For the analysis of the first 5 codons, dedicated A-site offset was individually estimated considering the short or absent 5′ UTR of mitochondrial mRNA (except MT-ATP6 and MT-ND4).

### Results

Due to the process of mitochondrial isolation, the final material is obtained only in a small amount, hampering robust library construction. Therefore, an original T7 high-resolution Thor-Ribo-Seq approach based on RNA-dependent RNA amplification was introduced. Application of this approach did not affect the codon-wise measurement of ribosome occupancy (FIG. 9, (b)). Therefore, MitoIP-Thor-Ribo-Seq was used for downstream analysis. Chloramphenicol used in this method has been proposed to cause context-selective inhibition of extension (favoring Ala/Ser and disfavoring Gly) at the second amino acid from the end (penultimate amino acid, or E-site) in bacteria (References 65 to 68 and Reference 34), but no such bias was observed in the mitochondrial data (FIG. 9, (c)).

Finally, compared with the published conventional MitoRibo-Seq (References 69 to 74), MitoIP-Thor-Ribo-Seq of the present invention showed comparable or better results in recovery of mitochondrial footprints (FIG. 10). Notably, the convenience of being able to isolate mitochondria from multiple samples in parallel and the low input library construction method allowed to examine mitochondrial translation in more than 60 samples in this study.

### Accession Number

The results of MitoIP-Thor-Ribo-Seq (GEO: GSE237154) obtained in this study have been deposited in the National Center for Biotechnology Information (NCBI) database. In this study, reported data of standard Ribo-Seq, RNA-Seq, and BRIC-Seq (GSE233555 and GSE233374, Reference 61) were also used.

### Example 4

### Application to Multiple Specimens

The Thor-Ribo-Seq of the present invention can be applied to multi-specimen analysis. In this example, the protocol for experiments using a 96-well plate is shown.

### Protocol for 96-Well Linear-Thor

### (1) Cell Lysis

Prepare cell samples for 96 wells.

Prepare lysis buffer for 96 wells according to the composition shown in the following table.

TABLE 2

| | | | Final concentration | |
|---|---|---|---|---|
| 1M Tris, pH 7.5 | 200 | μL | 20 | mM |
| 5M NaCl | 300 | μL | 150 | mM |
| 1M MgCl₂ | 50 | μL | 5 | mM |
| 0.1M DTT | 100 | μL | 1 | mM |
| 10% Triton X-100 | 1000 | μL | 1% | |
| H₂O | 8185 | μL | | |
| 2 Unit/μL Turbo Dnase I | 125 | μL | 0.025 | U/μL |
| *Chloramphenicol 34 mg/mL | 30 | μL | 100 | μg/mL |
| *Cycloheximide 100 mg/mL | 10 | μL | 100 | μg/mL |
| Total volume | 10000 | μL | | |

*Dnase I was added.

Rinse the cells in the 96 wells once with 180 μL of cold PBS on ice using an 8-well pipette, then fully absorb and discard PBS, and add 50 μL of lysis buffer. Perform the above operation all at once and repeat 12 times. Detach the cells by 3 times of pipetting and transfer them to a 0.2 μm plate (Corning (registered trademark) FiltrEX™ 96-well Filter Plate). Apply a gas permeable film (Greiner Bio-One 676051) and centrifuge the plate at 1500 g and 4° C. for 10 minutes to obtain cell lysate samples of 96 wells (about 40 μL). Seal the samples on the 96-well plate with an aluminum sealing film, freeze with liquid nitrogen and store them at −80° C. Confirm the concentration of some samples.

### (2) Nuclease Footprinting and Purification of Footprint Fragments

Dispense 2 μL of RNase I (10 U/μL) into each well of a 96-well plate, then add 36 μL of cell lysate sample, mix, and incubate them at 25° C. for 45 minutes. To terminate the RNase I reaction, place the plate on a cooling block. Prepare RNA inhibitor, I-well plate en™ SUPERase. In™ (1.33 of SUPERase In™ and 0.67 of lysis buffer for dilution) in tubes of 8-tube strip and dispense the cell lysate in a volume of 34 μL with a liquid dispensing robot ASSIST PLUS (INTEGRA Biosciences). Pass the RNaseI-treated cell lysates through a 96-well plate filled with Sepacryl S-400 HR (GE HealthCare, diluted 2-fold in the lysis buffer without Triton X-100) to purify them (800 g, 2 minutes, 4° C.). Purify RNA from the cell lysate using Direct-zol-96 RNA Kit (Zymo Research).

### (3) Preparation of Pre-Adenylated Linker

Prepare a mixture (not containing primers) for 96 wells according to the following table.

TABLE 3

| | 26.6 μL system | | Mixture for | |
|---|---|---|---|---|
| | (for 1 well) | | 100 wells | |
| 100 μM 5′ p-linker-ddC primer | 1.6 | μL | / | |
| 10x 5′ DNA Adenylation reaction | 2.7 | μL | 266 | μL |
| buffer | | | | |
| 1 mM ATP | 2.7 | μL | 266 | μL |
| Mth RNA Ligase | 2.7 | μL | 266 | μL |
| H₂O | 17.0 | μL | 1702.4 | μL |
| Total volume | 26.6 | μL | 2500.4 | μL |

On a LoBind PCR plate (Eppendorf twin.tec (registered trademark) PCR Plates LoBind (registered trademark)), dispense 25 μL each of the mixture (without primer) with an Eppendorf aliquoter 2.5 mL, then dispense 1.6 μL each of 100 M 5′ p-linker-ddC primer (dispensed on a separate 96-well plate) into each well, and incubate them at 65° C. for 1 hour, followed by 5 minutes at 85° C. Perform purification using Oligo Clean & Concentrator (Zymo research).

### (4) Disome Marker (50 to 80 nt) Purification

Purify 50 to 80 nt markers from the excised gels (if not available, start from the electrophoresis). Perform RNA extraction by ethanol precipitation using Dr. GenTLE Precipitation Carrier (Takara 9094).

### (5) First Dephosphorylation and Linker Ligation

Also ligate markers excised in 17 to 34 nt and 50 to 80 nt and purified. Set Thermo Mixer C (Eppendorf block incubator) at 95° C.

### <Dephosphorylation>

Treat the sample obtained in (2) above in Thermo Mixer C at 95° C. for 2 minutes and place it on ice for 3 minutes.

Treat markers prepared in tubes of 8-tube strip in a thermal cycler at 95° C. for 2 minutes and place them on ice for 3 minutes.

TABLE 4

| | | |
|---|---|---|
| 10 μM 34 nt RNA (NI-801*) | 0.5 | μL |
| 10 μM 26 nt RNA (NI-800*) | 0.5 | μL |
| 10 μM 17 nt RNA (SI-029) | 0.5 | μL |
| RNase Free water | 5.5 | μL |
| Total volume | 7 | μL |

*NI-801 and NI-800 are size markers described in Non-patent document 7.

50 to 70nt RNA 7 μL

Prepare a required volume of PNK mixture in 5 mL tubes according to the following table and manually dispense it in a volume of 3 μL each on Lobind PCR plate.

Eppendorf dispenser 0.5 ml for samples+excess 18 μL

TABLE 5

| | |
|---|---|
| x1 | 10² |

| RNA sample | 7 | μL | / | |
|---|---|---|---|---|
| 10x T4 PNK buffer | 1 | μL | 102.0 | μL |
| T4 PNK | 1 | μL | 102.0 | μL |
| SUPERase In | 1 | μL | 102.0 | μL |
| Total volume | 10 | μL | 306 | μL |

Dispense 7 μL of sample onto the Lobind PCR plate using the liquid dispensing robot ASSIST PLUS, mix, and incubate them at 37° C. for 1 hour.

### <Linker Ligation>

Manually dispense 9 μL of Ligation mixture onto the Lobind PCR plate.

TABLE 6

| | | |
|---|---|---|
| Ligation mixture | x1 | 10² |

| 50% PEG-8000 | 7 | μL | 714.0 | μL |
|---|---|---|---|---|
| 10x T4 RNA ligase buffer | 1 | μL | 102.0 | μL |
| Preadenylated linker (5 μM) | 1 | μL | / | |
| T4 Rnl2(tr) K227Q (200 U/μL) | 1 | μL | 102.0 | μL |
| Total volume | 10 | μL | 918 | μL |

Dispense 1 μL each of the preadenylated linker (5 μM) into Ligation mixture by using the ASSIST PLUS liquid dispensing robot. Transfer 10 μL of the dephosphorylated sample to the Ligation mixture using the liquid dispensing robot ASSIST PLUS, mix them, incubate at 22° C. for 3 hours, and store the mixture at 4° C.

### <D&L Deadenylase/Lambda Exonuclease Treatment>

Dispense 34 μL each of D&L mixture into tubes of 8-tube strip.

TABLE 7

| | | |
|---|---|---|
| D&L mixture | x1 | 136 |

| RNA sample | 20 | μL | / | |
|---|---|---|---|---|
| 5′ Deadenylase | 1 | μL | 136.0 | μL |
| Lambda exonuclease | 1 | μL | 136.0 | μL |
| Total volume | 22 | μL | 272.0 | μL |

Dispense the mixture in a volume of 2 μL each to samples. Incubate at 30° C. for 45 minutes and store the mixture at 4° C. To terminate ligation, dispense 66 μL of Trizol LS into each sample, mix them, collect the mixture in Invitrogen™ Phasemaker™ tube, and perform purification to extract RNA. Incubate the ligated 17 to 34 nt and 50 to 80 nt markers and the samples at 95° C. for 3 minutes on a heat block and place them on ice for 2 minutes. Electrophorese the reaction mixtures on Gellex 10% acrylamide gel at 10 mA for 60 minutes. Excise the portions of the markers.

(6) Perform Gel Purification after the First Ligation Using Invitrogen™ GlycoBlue™ Coprecipitant.
(7) Perform rRNA Depletion Using RiboPOOL Kit (siTOOLs Biotech).

### (8) Perform Bead Purification.

### (9) In Vitro Transcription

TABLE 8

| 20 μM SI194 | 0.5 | μL | 1.25 | μL |
|---|---|---|---|---|
| temp RNA with T7 linker | 7 | μL | / | |
| 10x T7-Scribe Transcription Buffer | 2 | μL | 5 | μL |
| 100 mM ATP | 1.5 | μL | 3.75 | μL |
| 100 mM GTP | 1.5 | μL | 3.75 | μL |
| 100 mM CTP | 1.5 | μL | 3.75 | μL |
| 100 mM UTP | 1.5 | μL | 3.75 | μL |
| 100 mM DTT | 2 | μL | 5 | μL |
| ScriptGuard Rnase Inhibitor | 0.5 | μL | 1.25 | μL |
| T7-Scribe Enzyme Solution | 2 | μL | 5 | μL |
| Total volume | 20 | μL | 32.5 | μL |

Dispense 13 μL each of sample, add a template, and incubate them at 37° C. for 2.5 hours. Add 1 μL of Invitrogen™ TURBO™ DNase and incubate the mixture at 37° C. for 20 minutes. Perform purification using RNAclean XP beads (Beckman Coulter Life Sciences).

### (10) Perform Gel Purification of IVT RNA.

### (11) Second Dephosphorylation and SI183 Linker Ligation

Transfer 8 μL (150 ng) each of samples to tubes of 8-tube strip, incubate at 95° C. for 2 minutes, and place them on ice for 3 minutes.

### <Linker Ligation>

TABLE 9

| | x1 | | 2.5 | |
|---|---|---|---|---|
| RNA sample | 8 | μL | / | |
| 10x T4 PNK buffer | 1 | μL | 2.5 | μL |
| SUPERase In | 1 | μL | 2.5 | μL |
| 50% PEG-8000 | 7 | μL | 17.5 | μL |
| 10x T4 RNA ligase buffer | 1 | μL | 2.5 | μL |
| SI183 Preadenylated linker (20 μM) | 1 | μL | 2.5 | μL |
| T4 Rnl2(tr) K227Q (200 U/μL) | 1 | μL | 2.5 | μL |
| Total volume | 20 | μL | 30 | μL |

Dispense 12 μL into the samples and incubate at 22° C. for 2.5 hours, and then store the mixture at 4° C.

### <Deadenylase/Lambda Exonuclease Treatment>

Dispense 34 μL each of D&L mixture into tubes of 8-tube strip.

TABLE 10

| | | |
|---|---|---|
| D&L mixture | x1 | 2.5 |

| RNA sample | 20 | μL | / | |
|---|---|---|---|---|
| 5′ Deadenylase | 1 | μL | 2.5 | μL |
| Lambda exonuclease | 1 | μL | 2.5 | μL |
| Total volume | 22 | μL | 5 | μL |

Dispense 12 μL each into the samples. Incubate at 30° C. for 45 minutes, and then store the mixture at 4° C. Perform purification using RNAclean XP beads.

### (12) Reverse Transcription Reaction

### <Reverse Transcription Reaction>

Prepare 10 μL each of samples in tubes of 8-tube strip, add 2 μL of 1.25 μM RT Primer SI186, incubate at 65° C. for 5 minutes, and then place the mixture on ice.

Prepare a mixture according to the following table, dispense 8 μL of it into the sample+RT primers, and incubate the mixture at 50° C. for 30 minutes.

TABLE 11

| | |
|---|---|
| x1 | 2.2 |

| | | | | |
|---|---|---|---|---|
| 5x Protoscript II buffer | 4 | μL | 8.8 | μL |
| 10 mM dNTPs | 1 | μL | 2.2 | μL |
| 10x DTT | 1 | μL | 2.2 | μL |
| SUPERase In | 1 | μL | 2.2 | μL |
| Protoscript II | 1 | μL | 2.2 | μL |
| Sample + RT primer | 12 | μL | | |
| Total volume | 20 | μL | 17.6 | μL |

Add 1 μL of Exonuclease I and incubate the mixture at 37° C. for 30 minutes. Fully mix 2 μL of 0.5 M EDTA with the mixture to terminate the reaction.

### <Hydrolyze RNA>

Add 2.6 μL each of 1 M NaOH, mix, and incubate the mixture at 70° C. for 20 minutes.

Perform purification using RNAclean XP beads.

### (13) PCR Amplification and Barcode Addition

Perform 6 or 8 cycles of the reaction using 100 ng of IVT RNA.

TABLE 12

| | 100 μL System | |
|---|---|---|
| 5x Phusion HF buffer | 20 | μL |
| 10 mM dNTPs | 2 | μL |
| *Diluted 10 μM* NI798 Fw primer | 5 | μL |
| *Diluted 10 μM* NI799 Rv primer | 5 | μL |
| cDNA template | 5 | μL |
| H₂O | 62 | μL |
| Phusion polymerase (2 U/μL) | 1 | μL |
| Total volume | 100 | μL |

Prepare each sample in 100 μL system, divide it into two tubes in a volume of 50 μL each, and try 6 or 8 cycles.

Perform reactions of PCR using a thermal cycler for each cycle.

| | | |
|---|---|---|
| 98° C. | 30 s | |
| 98° C. | 10 s | x6, x8 |
| 65° C. | 10 s | |
| 72° C. | 5 s | |
| 72° C. | 10 s | PAUSE POINT −20° C. |
| 4° C. | ∞ | |

Perform gel purification.

### (14) Size Confirmation

Confirm the size, concentration, and mol number using a microchip electrophoresis system for DNA/RNA analysis, MultiNA (Shimadzu Corporation).

### REFERENCES

Reference 14. Van Gelder, R. N. et al. Amplified RNA synthesized from limited quantities of heterogeneous cDNA. Proc. Natl. Acad. Sci. U.S.A 87, 1663-1667 (1990).
Reference 15. Eberwine, J. et al. Analysis of gene expression in single live neurons. Proc. Natl. Acad. Sci. U.S.A 89, 3010-3014 (1992).
Reference 16. Hoeijmakers, W. A. M., Bartfai, R., Francoijs, K.-J. & Stunnenberg, H. G. Linear amplification for deep sequencing. Nat. Protoc. 6, 1026-1036 (2011).
Reference 17. Chen, C. et al. Single-cell whole-genome analyses by Linear Amplification via Transposon Insertion (LIANTI). Science 356, 189-194 (2017).
Reference 18. Hashimshony, T., Wagner, F., Sher, N. & Yanai, I. CEL-Seq: single-cell RNA-Seq by multiplexed linear amplification. Cell Rep. 2, 666-673 (2012).
Reference 19. Hashimshony, T. et al. CEL-Seq2: sensitive highly-multiplexed single-cell RNASeq. Genome Biol. 17, 77 (2016).
Reference 20. Bartlett, D. A. et al. High-throughput single-cell epigenomic profiling by targeted insertion of promoters (TIP-seq). J. Cell Biol. 220, (2021).
Reference 21. Lake, B. B. et al. Integrative single-cell analysis of transcriptional and epigenetic states in the human adult brain. Nat. Biotechnol. 36, 70-80 (2018).
Reference 22. Kargapolova, Y., Levin, M., Lackner, K. & Danckwardt, S. sCLIP-an integrated platform to study RNA--protein interactomes in biomedical research: identification of CSTF2tau in alternative processing of small nuclear RNAs. Nucleic Acids Res. 45, 6074-6086 (2017).
Reference 23. Su, R. et al. Global profiling of RNA-binding protein target sites by LACE-seq. Nat. Cell Biol. 23, 664-675 (2021).
Reference 24. Milligan, J. F., Groebe, D. R., Witherell, G. W. & Uhlenbeck, O. C. Oligoribonucleotide synthesis using T7 RNA polymerase and synthetic DNA templates. Nucleic Acids Res. 15, 8783-8798 (1987).
Reference 25. Arnaud-Barbe, N., Cheynet-Sauvion, V., Oriol, G., Mandrand, B. & Mallet, F. Transcription of RNA templates by T7 RNA polymerase. Nucleic Acids Res. 26, 3550-3554 (1998).
Reference 26. Conrad, T., Plumbom, I., Alcobendas, M., Vidal, R. & Sauer, S. Maximizing transcription of nucleic acids with efficient T7 promoters. Commun Biol 3, 439 (2020).
Reference 27. Dana, A. & Tuller, T. Determinants of translation elongation speed and ribosomal profiling biases in mouse embryonic stem cells. PLOS Comput. Biol. 8, e1002755 (2012).
Reference 28. Hussmann, J. A., Patchett, S., Johnson, A., Sawyer, S. & Press, WH. Understanding biases in ribosome profiling experiments reveals signatures of translation dynamics in yeast. PLOS Genet. 11, e1005732 (2015).
Reference 29. Weinberg, D. E. et al. Improved ribosome-footprint and mRNA measurements provide insights into dynamics and regulation of yeast translation. Cell Rep. 14, 1787-1799 (2016).
Reference 30. O'Connor, P. B., Andreev, D. E. & Baranov, P. V. Comparative survey of the relative impact of mRNA features on local ribosome profiling read density. Nat. Commun. 7, 12915 (2016).
Reference 31. Zhang, S. et al. Analysis of ribosome stalling and translation elongation dynamics by deep learning. Cell Syst 5, 212-220.e6 (2017).
Reference 32. Dao Duc, K. & Song, Y. S. The impact of ribosomal interference, codon usage, and exit tunnel interactions on translation elongation rate variation. PLOS Genet. 14, e1007166 (2018).
Reference 33. Tunney, R. et al. Accurate design of translational output by a neural network model of ribosome distribution. Nat. Struct. Mol. Biol. 25, 577-582 (2018).
Reference 34. Mohammad, F., Green, R. & Buskirk, A. R. A systematically-revised ribosome profiling method for bacteria reveals pauses at single-codon resolution. Elife 8, e42591 (2019).
Reference 35. Sharma, A. K. et al. A chemical kinetic basis for measuring translation initiation and elongation rates from ribosome profiling data. PLOS Comput. Biol. 15, e1007070 (2019).
Reference 36. Han, P. et al. Genome-wide survey of ribosome collision. Cell Rep. 31, 107610 (2020).
Reference 37. Gerashchenko, M. V. & Gladyshev, V. N. Ribonuclease selection for ribosome profiling. Nucleic Acids Res. 45, e6 (2017).
Reference 38. Liu, B. et al. Optimization of ribosome profiling using low-input brain tissue from fragile X syndrome model mice. Nucleic Acids Res. 47, e25 (2019).
Reference 39. Martinez, T. F. et al. Accurate annotation of human protein-coding small open reading frames. Nat. Chem. Biol. 16, 458-468 (2020).
Reference 40. Becker, A. H., Oh, E., Weissman, J. S., Kramer, G. & Bukau, B. Selective ribosome profiling as a tool for studying the interaction of chaperones and targeting factors with nascent polypeptide chains and ribosomes. Nat. Protoc. 8, 2212-2239 (2013).
Reference 41. Galmozzi, C. V., Merker, D., Friedrich, U. A., Doring, K. & Kramer, G. Selective ribosome profiling to study interactions of translating ribosomes in yeast. Nat. Protoc. 14, 2279-2317 (2019).
Reference 42. Archer, S. K., Shirokikh, N. E., Beilharz, T. H. & Preiss, T. Dynamics of ribosome scanning and recycling revealed by translation complex profiling. Nature 535, 570-574 (2016).
Reference 43. Shirokikh, N. E., Archer, S. K., Beilharz, T. H., Powell, D. & Preiss, T. Translation complex profile sequencing to study the in vivo dynamics of mRNA-ribosome interactions during translation initiation, elongation and termination. Nat. Protoc. 12, 697-731 (2017).
Reference 44. Ichihara, K. et al. Combinatorial analysis of translation dynamics reveals eIF2 dependence of translation initiation at near-cognate codons. Nucleic Acids Res. 49, 7298-7317 (2021).
Reference 45. Wagner, S. et al. Selective translation complex profiling reveals staged initiation and co-translational assembly of initiation factor complexes. Mol. Cell 79, 546-560.e7 (2020).
Reference 46. Bohlen, J., Fenzl, K., Kramer, G., Bukau, B. & Teleman, A. A. Selective 40S footprinting reveals cap-tethered ribosome scanning in human cells. Mol. Cell 79, 561-574.e5 (2020).
Reference 47. Wagner, S. et al. Selective footprinting of 40S and 80S ribosome subpopulations (Sel-TCP-seq) to study translation and its control. Nat. Protoc. 17, 2139-2187 (2022).
Reference 48. Van Nostrand, E. L. et al. A large-scale binding and functional map of human RNA-binding proteins. Nature 583, 711-719 (2020).
Reference 49. Chen, S., Zhou, Y., Chen, Y. & Gu, J. fastp: an ultra-fast all-in-one FASTQ preprocessor. Bioinformatics 34, 1884-1890 (2018).
Reference 50. Dobin, A. et al. STAR: ultrafast universal RNA-seq aligner. Bioinformatics 29, 15-21 (2013).
Reference 51. Smith, T., Heger, A. & Sudbery, I. UMI-tools: modeling sequencing errors in Unique Molecular Identifiers to improve quantification accuracy. Genome Res. 27, 491-499 (2017).
Reference 52. Morales, J. et al. A joint NCBI and EMBL-EBI transcript set for clinical genomics and research. Nature 604, 310-315 (2022).
Reference 53. LUTHOR 3′ mRNA-Seq Library Prep Kit User Guide (https://www.lexogen.com/wp-content/uploads/2020/10/143UG290V0100_LUTHOR-3-mRNA-Seq-Kit_2020-10-21.pdf).
Reference 54. Taisei Wakigawa et al., Gravitational and mechanical forces drive mitochondrial translation through the cell adhesion-FAK axis. BioRxiv 2023.01.18.524628; doi: https://doi.org/10.1101/2023.01.18.524628.
Reference 55. Sasagawa, Y., Danno, H., Takada, H. et al. Quartz-Seq2: a high-throughput single-cell RNA-sequencing method that effectively uses limited sequence reads. Genome Biol 19, 29 (2018). 1710207991512_7.
Reference 56. Nakano, S. et al. NSUN3 methylase initiates 5-formylcytidine biogenesis in human mitochondrial tRNAMet. Nat. Chem. Biol. 12, 546-551 (2016).
Reference 57. Kawarada, L. et al. ALKBHI is an RNA dioxygenase responsible for cytoplasmic and mitochondrial tRNA modifications. Nucleic Acids Res. 45, 7401-7415 (2017).
Reference 58. Asano, K. et al. Metabolic and chemical regulation of tRNA modification associated with taurine deficiency and human disease. Nucleic Acids Res. 46, 1565-1583 (2018).
Reference 59. Suzuki, T. et al. Complete chemical structures of human mitochondrial tRNAs. Nat. Commun. 11, 4269 (2020).
Reference 60. Lin, H. et al. CO2-sensitive tRNA modification associated with human mitochondrial disease. Nat. Commun. 9, 1-17 (2018).
Reference 61. Tomuro, K., et al. Absolute calibration of ribosome profiling assesses the dynamics of ribosomal flux on transcripts. bioRxiv (2023) doi: 10.1101/2023.06.20.545829.
Reference 62. Kashiwagi, K. et al. eIF2B-capturing viral protein NSs suppresses the integrated stress response. Nat. Commun. 12, 7102 (2021).
Reference 63. Dobin, A. et al. STAR: ultrafast universal RNA-seq aligner. Bioinformatics 29, 15-21 (2013).
Reference 64. Smith, T., Heger, A. & Sudbery, I. UMI-tools: modeling sequencing errors in Unique Molecular Identifiers to improve quantification accuracy. Genome Res. 27, 491-499 (2017).
Reference 65. Marks, J. et al. Context-specific inhibition of translation by ribosomal antibiotics targeting the peptidyl transferase center. Proc. Natl. Acad. Sci. U.S.A 113, 12150-12155 (2016).
Reference 66. Syroegin, E. A. et al. Structural basis for the context-specific action of the classic peptidyl transferase inhibitor chloramphenicol. Nat. Struct. Mol. Biol. 29, 152-161 (2022).
Reference 67. Tsai, K. et al. Structural basis for context-specific inhibition of translation by oxazolidinone antibiotics. Nat. Struct. Mol. Biol. 29, 162-171 (2022).
Reference 68. Shichino, Y. & Iwasaki, S. Compounds for selective translational inhibition. Curr. Opin. Chem. Biol. 69, 102158 (2022).
Reference 69. Rooijers, K., Loayza-Puch, F., Nijtmans, L. G. & Agami, R. Ribosome profiling reveals features of normal and disease-associated mitochondrial translation. Nat. Commun. 4, 2886 (2013).
Reference 70. Pearce, S. F. et al. Maturation of selected human mitochondrial tRNAs requires deadenylation. Elife 6, e27596 (2017).
Reference 71. Morscher, R. J. et al. Mitochondrial translation requires folate-dependent tRNA methylation. Nature 554, 128-132 (2018).
Reference 72. Li, S. H.-J., Nofal, M., Parsons, L. R., Rabinowitz, J. D. & Gitai, Z. Monitoring mammalian mitochondrial translation with MitoRiboSeq. Nat. Protoc. 16, 2802-2825 (2021).
Reference 73. Schöller, E. et al. Balancing of mitochondrial translation through METTL8-mediated m3C modification of mitochondrial tRNAs. Mol. Cell 81, 4810-4825.e12 (2021).
Reference 74. Soto, I. et al. Balanced mitochondrial and cytosolic translatomes underlie the biogenesis of human respiratory complexes. Genome Biol. 23, 170 (2022).

1. A method for preparing a DNA library from a sample containing RNA, comprising:
(1) ligating a first DNA linker to the 3′ end of at least one RNA molecule in a sample containing RNA to obtain an RNA molecule having the first DNA linker, wherein the first DNA linker comprises at least a promoter antisense sequence;
(2) hybridizing a first oligonucleotide with the RNA molecule having the first DNA linker to obtain the RNA molecule having a double-stranded DNA promoter region, wherein the first oligonucleotide comprises a promoter sense sequence;
(3) obtaining a complementary strand of the RNA molecule having the first DNA linker (complementary RNA) by in vitro transcription using the RNA molecule having the double-stranded DNA promoter region as a template and an RNA polymerase that recognizes the promoter sequence and initiates transcription; and
(4) ligating a second DNA linker to the 3′ end of the complementary RNA and further obtaining a single-stranded DNA by reverse transcription using the complementary RNA having the second DNA linker as a template.

2. The method according to claim 1, which further comprises (5) obtaining a double-stranded DNA by PCR using the single-stranded DNA obtained in (4) as a template.

3. The method according to claim 1, wherein the RNA polymerase used in (3) is T7 RNA polymerase.

4. The method according to claim 1, wherein the first DNA linker and the first oligonucleotide contain a sequence that increases transcription efficiency in a flanking region of the promoter sequence.

5. The method according to claim 1, wherein the DNA library is a next generation sequencing cDNA library.

6. The method according to claim 1, wherein the sample containing RNA is a lysate of less than 1×10⁶ cells.

7. The method according to claim 1, which is performed to generate a cDNA library for ribosome profiling.

8. The method according to claim 7, wherein the at least one RNA molecule in the sample containing RNA is a ribosome footprint, the 3′ end of which is dephosphorylated prior to (1).

9. The method according to claim 1, which is for analyzing multiple samples using a multi-well plate.

10. The method according to claim 1, which is for analyzing mitochondrial translation.

11. A method for sequencing and/or quantifying an RNA molecule in a sample containing RNA, comprising
performing (1) to (4) described in claim 1 to prepare a DNA library; and
analyzing the DNA library by a next-generation sequencing method to obtain data of sequencing and/or quantifying the RNA molecule in the sample containing RNA.

12. A kit for performing the method according to claim 1, comprising
a first DNA linker comprising at least a promoter antisense sequence,
a first oligonucleotide comprising at least a promoter sense sequence; and
a second DNA linker.

13. The kit according to claim 12, which further comprises
an RNA polymerase that recognizes the promoter sequence; and/or
a reverse transcriptase; and/or
a second oligonucleotide used as a primer for reverse transcription; and/or
a pair of primer oligonucleotides for PCR.

14. The kit according to claim 12, wherein the first DNA linker comprises any of the following nucleotide sequences:
(SEQ ID NO: 1)
AGATCGGAAGAGCACACGTCTGAACTCCAGTCACATTATCCCTATAGTGA
GTCGTATTAAATTC
and
(SEQ ID NO: 2)
AGATCGGAAGAGCACACGTCTGAACTCCAGTCACCCTATAGTGAGTCGTA
TTAGTCA.

15. The kit according to claim 12, wherein the first oligonucleotide comprises any of the following nucleotide sequences:
(SEQ ID NO: 3)
TGACTAATACGACTCACTATAGGGTGACTGGAGTTCAG
and
(SEQ ID NO: 4)
GAATTTAATACGACTCACTATAGGGATAATGTGACTGGAGTTCAG.

16. The kit according to claim 12, wherein the second DNA linker comprises the following nucleotide sequence;

| (SEQ ID NO: 5) | |
|---|---|
| | AGATCGGAAGAGCGTCGTGTAGGGAAAGAG |

17. The kit according to claim 12, wherein the first DNA linker and/or the second DNA linker further comprises a sequence for a unique molecular identifier (UMI) and/or a sample identifier sequence.
